(19) United States
(12) Patent Application Publication
(10) Pub. No.: US 2023/0383264 A1
(43) Pub. Date: Nov. 30, 2023

(54) FULLY FUNCTIONAL CONTRACTILE GASTROINTESTINAL ORGANOID SYSTEM, GENERATED FROM HUMAN-INDUCED PLURIPOTENT STEM CELLS TO DEVELOP A HIGH THROUGHPUT SYSTEM FOR DETECTING EXISTING AND NEWLY-EMERGING PATHOGENS, DRUGS, TOXICITY AND THEREOF

(71) Applicant: NeyroblastGX LLC,, Murrieta, CA (US)

(72) Inventors: Aynun N. Begum, Murrieta, CA (US); Alexander V. Michkov, Murrieta, CA (US)

(21) Appl. No.: 18/324,091

(22) Filed: May 25, 2023

Related U.S. Application Data
(60) Provisional application No. 63/365,328, filed on May 25, 2022.

(51) Int. Cl. C12N 5/071; C12N 5/077; G01N 27/02

(52) U.S. Cl. CPC C12N 5/0697; C12N 5/0679; C12N 5/0656; C12N 5/0661; G01N 27/02; C12N 2506/45; C12N 2501/16; C12N 2501/415; C12N 2501/119; C12N 2501/11; C12N 2501/998; C12N 2513/00; C12N 2533/72

(57) ABSTRACT

A gastrointestinal organoid provides a fully functional complex GIO consisting of epithelial, endothelial, and mesenchymal cells with the microbiome of a natural human gastrointestinal system and exhibiting contractile behavior, useful for the rapid and sensitive screening of pathogens, toxins, drugs, environmental factors, and other compounds or diseases.

### FIELD OF THE INVENTION

This invention relates to the optimized contractile gastrointestinal organoids from human induced pluripotent stem cells (HiPSCs) combined with a high-throughput electroconductive plate for rapid screening of pathogens, drugs, toxicity, and other purposes.

### BACKGROUND

The global mobility of humans, non-human animals, plants, and products has substantially increased over time, which drives infectious diseases to new locations.^{1,2} In recent years, the 2019 coronavirus pandemic (COVID-19) and related viral infections have traveled rapidly due to their easy access from one place to another.^{2,3} These events affected the US and global healthcare system, and people still suffer from COVID, organ degeneration, and mortality. In addition, the global population is increasing, causing pathogens to likely develop resistance to current treatments. Acute infectious gastroenteritis is one of the common illnesses observed around the world. 4 Norovirus has become the most frequent cause of viral gastroenteritis in the United States (US), responsible for 19 to 21 million total illnesses annually.^{5,6} It is estimated to cause 56,000-71,000 hospitalization and 570-800 death annually in the US. Due to its environmental stability, Norovirus is currently implicated in nearly 50% of all foodborne outbreaks. 6 Thus, we need prevention strategies to eliminate or reduce new and existing pathogen threats. So, in this context, fully-functional gastrointestinal organoids (GIO)s from human induced pluripotent stem cells (HiPSCs) mimic the complete human system and can provide a unique opportunity to study food and environmental pathogens, the microbiome, and related toxicity to prevent acute and chronic viral, bacterial, and parasitic infections.

HiPSCs-derived human intestine, gut, and intermediatory GIO or fully functioning GIOs have been reported in various publications for studying gastroenteritis, celiac, Crohn's, and many other intestine-related diseases. 7,8,9,10 But none of these publications reports a fully functional complex GIO consisting of epithelial, endothelial, and mesenchymal cells and maintaining a natural (i.e., present in the natural human gastrointestinal system) microbiome with contractile behavior. Intestinal contractile behavior means peristaltic contractility, a crucial requirement for normal digestive tract function that moves foods through the digestive tract. This contractile behavior achievement in the reproducible organoid of the invention marks a pivotal advancement in recapitulating human intestinal physiological systems in real-time to accurately study diseases and other conditions and characteristics of the gastrointestinal system.

Unlike reported intestinal organoids, the contractile GIO of the present invention is derived from HiPSCs-derived gut spheroids. Spheroids are cell aggregates, self-assembling in an environment that prevents attachment to a flat surface. When these 3D spheroids are treated according to the invention with Activin A (100 ng/mL), Wnt3A, and FGF4 (50 ng/mL), the gut spheroids appear, a polarized columnar epithelium with villi and crypts patterning, which are called gut spheroids. These gut spheroids are removed from the culture system and seeded on a gel matrix dome in an intestinal medium containing Rspondin (100 ng/mL), Noggin (100 ng/mL), and EGF (200 ng/mL), and enables the production of contractile organoids, which recapitulate human physiology and cytoarchitecture to hold a complete microbiome system. Indeed, a culture method is needed to produce a complex, contractile, and complete GIO with a high throughput micro-physiological system. In this context, the unique GIO culture system of the present invention mimics the human micro-physiological system combined with a high throughput electroconductive plate ready to test for any GI disease of interest. The present invention is believed to be the first optimized GIO with an electroconductive plate and a kit readily available for testing environmental pathogen interaction with the microbiome, gastroenteritis, and gut inflammation. This model is not limited to environmental pathogens, but can also be used to test for colon polyps, cancer, and multiple degeneration-related intestine and immune systems.

### SUMMARY OF THE INVENTION

The present invention relates to a fully functional mature GIO generated from HiPSC-derived 3D gut spheroids, a new and novel approach for GIO development. The GIO of the invention has tissue complexity of the human intestinal epithelial structures surrounded, especially completely surrounded, by stratified mesenchyme, which contains muscle cells and sub-epithelial fibroblasts. The GIO system of the invention can produce mature intestinal phenotypes within 18 days, expressing restricted crypts (Ki67⁺), goblet cells (MUC2⁺), Paneth cells (lysozyme⁺), endocrine cells (PDX11, and enteroendocrine cells (chromogranin A⁺) compared to the reported systems that require over 30 days.^{7, 11, 12} The GIO with these specialized intestinal cells can transport protein and have anti-microbial action, which typically takes a long time, more than 6-12 weeks, to generate¹¹. But the GIO of the invention only requires 3 weeks to be generated. Therefore, the GIO system of the invention is more rapid and cost-effective and can be produced on a robust scale.

The present invention, therefore, has at least the following important unique, and first-ever aspects:
a. The GIO of the invention derives from gut spheroid cells.
b. The GIO of the invention is fully functionally contractile with motile ability.
c. The GIO of the invention has been proven by testing (with bacterial endotoxin and microbiota to maintain the microbiome which is comparable or the same as that of an in vivo human gastrointestinal system.
d. The GIO of the invention can be grown on a gel matrix dome on a multi-well electroconductive ECIS plate to provide an electrical signal (nanofarad or resistance), whereby the GIO can be used to test and evaluate the effects of pathogens or drugs on a treated GIO. The high throughput plates (8, 16, 96, or 384 well plates containing electrodes) can be utilized for rapid and sensitive screening of various pathogens, toxins, drugs, environmental factors, and other compounds.

In one embodiment, the invention relates to a gastrointestinal organoid which comprises:
gastrointestinal epithelial and endothelial cells;
a stratified mesenchyme surrounding said epithelial and endothelial cells and which contains muscle cells and sub-epithelial fibroblasts; and
wherein said gastrointestinal organoid exhibits fully functional contractile behavior.

In another embodiment, the gastrointestinal organoid further comprises smooth muscle cells, an enteric nervous system, and interstitial cells of Cajal.

In another embodiment, the gastrointestinal organoid comprises muscle cells, wherein the muscle cells comprise smooth muscle cells, fibroblasts, and myofibroblasts.

In another embodiment, the gastrointestinal organoid according to the invention expresses restricted crypts (Ki67⁺), goblet cells (MUC2⁺), Paneth cells (lysozyme⁺), endocrine cells (PDX1⁺), and enteroendocrine cells (chromogranin A⁺).

In a further embodiment, the cells of the organoid are positioned on a gel-matrix dome.

In a further embodiment, the gel-matrix is comprised of a mixture gel matrix of acylated chitosan and Matrigel (Corning) or Geltrex (Invitrogen).

In a further embodiment, the gel matrix domes comprising said cells of said organoids are positioned in the wells of a multi-well plate, wherein the plate can be an electroconductive multi-well plate.

In a further embodiment, the organoids are further combined with a gut microbiome that resembles or is the same as that of an in vivo human gastrointestinal system.

In another embodiment, the invention comprises a method for preparing a gastrointestinal organoid comprising:
a) culturing human induced pluripotent stem cells (HiPSCs) in a first culture media to produce 3-dimensional gut spheroid cells;
b) treating said gut spheroid cells with Activin A;
c) treating said gut spheroid cells with Wnt3A and fibroblast growth factor 4 to produce large gut spheroid cells having a diameter of greater than about 200 μm;
d) seeding said large gut spheroids on a dome-shaped gel-matrix in intestinal media containing Rspondin, Noggin, and EGF; and
e) culturing said domed-shaped gel matrix with intestinal media comprising large gut spheroids to provide a complete gastrointestinal organoid.

In another embodiment, the method further comprises seeding said large gut spheroids on a dome-shaped gel matrix in wells of an electroconductive multi-well plate.

In a further embodiment, the treatment with Activin A is initiated on Day 3 from the beginning of said culturing of the human induced pluripotent stem cells (HiPSCs).

In another embodiment of the invention, the Wnt3A and FGF4 are added to the spheroids at Day 6 from the beginning of said culturing of the human induced pluripotent stem cells (HiPSCs).

In a further embodiment of the invention, the Rspondin is added to the spheroids while in a gel matrix dome at Day 11 from the beginning of said culturing of the human induced pluripotent stem cells (HiPSCs).

The invention further relates to a gastrointestinal organoid obtained by the methods described above.

In another embodiment, the invention relates to a method for determining the effect of a factor of interest on the human gastrointestinal system, with comprises exposing a gastrointestinal organoid as described above with a factor of interest and measuring current impedance by electric cell-substrate impedance sensing.

In a further embodiment, the invention relates to a method for determining the effect of a factor on the human gastrointestinal system, which comprises:
a) exposing a first portion of gastrointestinal organoids of claim 6 comprising electrodes in said wells of said electroconductive wells with a factor of interest,
b) measuring the current or impedance of current across said electrodes in the said first portion of said gastrointestinal organoids.

In a further embodiment, the method of the invention further comprises:
not exposing a second portion of the gastrointestinal organoids comprising electrodes in said wells of said electroconductive wells with the said factor of interest, measuring the current or impedance of current across said electrodes in said second portion of said gastrointestinal organoids; and
comparing the current or impedance of current across said electrodes in said first portion of said gastrointestinal organoids with the current or impedance of current across said electrodes in said second portion of said gastrointestinal organoids.

In another embodiment of the invention, the factor to be analyzed is a pathogen, drug, or environmental factor.

In a further aspect of the invention, the effect to be measured is the toxicity of said factor.

In another embodiment, the invention relates to a kit comprising a vial containing the organoid described above and an intestinal organoid maintenance medium.

In another embodiment of the kit of the invention, the organoid is positioned on a gel-matrix dome.

In a still further embodiment of the kit of the invention, the organoid is positioned in wells of a multi-well electroconductive plate.

### BRIEF DESCRIPTION OF THE DRAWINGS

FIG. 1 shows a time course of mature gastrointestinal organoids (GIO) development.

FIG. 2 shows the morphogenesis of posterior endoderm and hindgut.

FIG. 3 shows the formation of HiPSC-derived fully functional GIO.

FIG. 4 shows the formation of contractile GIO with marker expressions.

FIG. 5 shows susceptible (Sus) GIO develop to study pathogen infection.

FIG. 6 shows the effect of chronic LPS induction on TJP1 (tight junction protein 1).

FIG. 7 shows pathogen versus non-pathogenic bacterial response with 10-plex cytokine array.

FIG. 8 shows intestinal tight intracellular junction disruption by the pathogen.

FIG. 9 shows intestinal barrier defects induce paracellular permeability and leaking by the pathogen.

FIG. 10 shows microbiota enables a complete GIO system developed as a human in vivo.

FIG. 11 shows shotgun metagenomics to determine the proportional distribution of bacteria changes in the heatmap.

FIG. 12 shows the cluster graph of LPS versus non-LPS showed significant alteration of the bacterial community.

FIG. 13 shows beta diversity (Unifrac distance) analysis color-coded using various groups with treatment conditions.

FIG. 14 shows dominant bacterial families change with LPS.

FIG. 15 shows that GIO can hold in vivo human microbiomes with microbiota.

FIG. 16 shows fresh versus Cryo-GIO viability and stability.

FIG. 17 shows fresh versus Cryo-GIO stability to secrete cytokines after pathogen infection.

FIG. 18 shows fresh versus Cryo-GIO on TJP stability with or without pathogens.

FIG. 19 shows high throughput (HT) electroconductive sensing array prototype development for GIO signaling.

FIG. 20 shows high throughput (HT) electroconductive sensing array development for rapid screening of fresh versus Cryo-GIO.

FIG. 21 shows the correlation of HT electroconductive signal with tight junction protein of GIO.

### DETAILED DESCRIPTION OF THE INVENTION

The present invention will now be described hereinafter with reference to the accompanying drawings and examples, in which embodiments of the invention are shown. This description is not intended to be a detailed catalog of all the different ways the invention may be implemented or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which does not depart from the instant invention.

Hence, the following descriptions are intended to illustrate some particular embodiments of the invention and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is to describe particular embodiments only and is not intended to be limiting to the invention.

### I. Definitions

As used in the description of the invention and the appended claims, the singular forms “a,” “an,” and “the” are intended to include the plural forms as well unless the context indicates otherwise.

The term “HiPSCs” refers to human induced pluripotent stem cells with similar phenotypical and genotypical characteristics as human embryonic stem cells (hESCs) or human pluripotent stem cells (HPSCs). Human somatic cells have been reprogrammed directly to pluripotency by ectopic expression of four transcription factors (Oct4, Sox2, Klf4, and Myc) to yield HiPSCs. HiPSCs have self-renewing capabilities similar to hESCs and can undergo three germ layers, producing all the germ layer cells with appropriate growth factors such as endodermal lineage-derived intestinal cells or organoids.

The term “organoid” or “organoids” is a singular or plural form used to express due to their use in a certain cell culture environment or to perform a specific assay. Organoids are tiny (mm levels), self-organized, three-dimensional tissue cultures that are derived from pluripotent stem cells, which can be derived from mice, primates, or other human stem cells such as mesenchymal or bone marrow-derived stem cells. Methods of producing organoids such as intestinal organoids can be found in U.S. Pat. Nos. 9,719,068 and 10,174,289, and PCT Publications WO 2016/061464 and WO 2018/106628, each of which is hereby expressly incorporated by reference in its entirety. In the present invention, the inventors developed organoids from HiPSCs. These human stem cell-based tissue cultures can be created to replicate similar complexity of human organs with a certain type of cells or tissues. The present invention importantly generated organoids to recapitulate human organs with collective cell types to perform particular functions like the intestine.

As used herein, the term “GIO” refers to the gastrointestinal organoids generated from HiPSCs with various endodermal and Mid/Hindgut growth factors such as human recombinant activin A, Wnt3A, FGF4, R-spondin 1 (Rspondin), noggin and epidermal growth factor (EGF). The inventors refer to intestinal organoids as gastrointestinal organoids (GIO) since the inventors generated a complete architecture of the human intestine and contained a luminal cavity bounded by epithelial layers and resembles sterile neonatal tissue. The inventors found the morphogenesis of the GIO of the invention includes microscopic brush borders, villus, and crypt-type structures. Besides, the inventors have observed intestine has highly convoluted tissues such as epithelium, endothelial, and mesenchyme, including absorptive enterocytes and the major secretory lineages cells such as goblet, Paneth, and enteroendocrine cells with motile behavior, which is only possible to obtain in the complete human embryonic gastrointestinal system. The GIO of the invention is a novel human intestinal system reported to contain complex and convoluted contractile tissue for the human model feature to maintain complete microbiome interaction, which has not been observed or found in any other model system, as referenced above. 7, 10,11,12,13

As used herein, “Immunocytochemistry” or “ICC” refers to an assay to determine the presence of a specific hind/midgut and intestinal protein or antigen expression in the GIO of the invention used by an antibody and visualization by the microscope. To validate the GIO functionality, the inventors performed ICC to determine prominent human intestinal markers such as SOX17⁺, FOXA2⁺, CDX2⁺, Ki67⁺, MUC2⁺, MUC6⁺, lysozyme⁺, PDX1⁺, chromogranin A⁺, Zo1⁺, SmoothA⁺, Map2⁺, GFAP⁺, E-cad⁺, and β-actin⁺. The inventors have ensured our GIO was viable by determining 4′,6-diamidino-2-phenylindole (DAPI), a positive area of the GIO marker cell. The GIO upregulated all the functional proteins and expressed over 80-90%. The inventors have used all the functional proteins of the intestine to establish that the GIO of the invention is a fully functional, complete, and mature intestine as human in vivo.

As used herein, the terms “High Throughput” or “HT,” is a method to describe the organoid system of the invention with an electroconductive plate that can be used to determine various pathogens with a small amount of time, with high sensitivity and in a non-invasive. The invention electroconductive plate with optimized GIO can screen over 50 drugs or pathogens in just 3-hour time points and can replace the work of a 3-month animal study, which is part of the novelty of the method of the invention.

As used herein, the term “optimized” refers to the organoid system of the invention with the same size grown on the defined geometrical electrode with the same media and gel matrix will lead us to no batch-to-batch variation. The GIO culture system of the invention is a unique, optimized organoid culture development as proposed for screening pathogens and toxicity.

As used herein, “contractile” refers to the innovative GIO of the invention with movement similar to a human in vivo system. Muscle contractions of GIO automatically move foods and fluids forward in humans or animals until they exit the anus or urethra. The GIO of the invention has the smooth muscle cells (SMC), enteric nervous system (ENS), and interstitial cells of Cajal (ICJ) appearing all in one system and coordinating for contractions 14,15, as stated here.

As used herein, “innate immune,” or “Prof,” refers to regulating the immune response to pathogen infection or inflammation via bacterial endotoxin LPS used for our system to determine proinflammatory cytokine (ProC) release. The inventors have determined various cytokines after LPS induction, such as IL-2, IL-6, IL-8, IL-10, IL-13, MIP-1a, MIP-113, TNF-α, IL-U, IFNγ in our GIO system. IL-2, IL-6, IL-8, TNF-α, MIP-1α, and MIP-1β upregulated within 24 h but IL-10, IL-1β, and IFNγ were upregulated when treated with only 100 μg/mL LPS for 48 h. These cytokine secretions are critical for innate immune signaling, which the GIO of the invention is able to recreate like a human gut immune system.

As used herein, “TJP,” tight junction protein is an important marker for the paracellular damage and leaking of the gut as observed in the GIO of the invention after induction of LPS or pathogen. To study TJP disruption, the inventors used “lucifer yellow dye,” or “LuciY,” to observe the leaking gut via disrupting tight junction by LPS or pathogen. The LuciY entrance to the GIO core proves leaky gut is a similar occurrence observed in the human intestine in vivo by pathogen invasion.

As used herein, “Fresh-GIO” and “Cryo-GIO” refer to GIO maintaining in standard culture conditions (37° C. with 5% CO 2) are the Fresh-GIO. But the inventors called it Cryo-GIO when the inventors collected Fresh-GIO in cryopreserving media and cooling in liquid N2 (LN2) to store and bring it again to standard culture condition. Fresh-GIO was never exposed to cold conditions, but Cryo-GIO was exposed once or twice in LN2. The inventors verified Cyro-GIO is stable in the freeze and thaw cycle and recapitulate the same human in vivo functionality as Fresh-GIO.

As used herein, “mesenchyme” is a type of loosely organized animal embryonic connective tissue of undifferentiated cells that give rise to most tissues like epithelium which is formed of columnar absorptive cells with a striated border, many goblet cells, endocrine cells, and basal stem cells but no Paneth cells. A stratified epithelium is a type of epithelial tissue composed of more than one layer of epithelial cells.

As used herein, “shotgun metagenomics is a technique used to sequence the genomes of entire microbial communities. Shotgun metagenomic sequencing is an alternative approach to studying uncultured microbiota that avoids these limitations. Shotgun metagenomic sequencing allows researchers to comprehensively sample all genes in all organisms present in a given complex sample. The method enables microbiologists to evaluate bacterial diversity and detect the abundance of microbes in various environments. Shotgun metagenomics also provides a means to study unculturable microorganisms that are otherwise difficult or impossible to analyze. (see Illumina, Illuminca.com, on Shotgun Metagenomic Sequencing; and Quince et al., Shotgun metagenomics, from sampling to sequence and analysis, 2017, https://aura.abdn.ac.uk/bitstream/2164/10167/1/NBT_R37313C_Iine_edit_S.1_1493899923_1_final_for self_archiving.pdf and Usyk et al., Comprehensive evaluation of shotgun metagenomics, amplicon sequencing, and harmonization of these platforms for epidemiological studies, CellReports Methods 3,100391 Jan. 23, 2023).

As used herein, “differentiate” or “differentiated” are used to refer to the process and conditions by which immature (unspecialized) cells acquire characteristics, becoming mature (specialized) cells, thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or a subset of cell types, and cannot, under normal circumstances, differentiate into a different cell type or revert to a less differentiated cell type.

Features of the GIO of the invention.

The novel human GIO of the invention can contract naturally, which is the first reported system without a specific oxygen chamber or air-liquid interface. 13 It is important to note that the previously reported specific air-liquid chamber model derived contractile organoids derived from mice tissue, not from human tissue. To date, the inventors are not aware of any reported method to generate a human contractile intestine from HiPSCs. 14 Moreover, motile gut generation is highly complex if smooth muscle cells (SMC), the enteric nervous system (ENS), and the interstitial cells of Cajal (ICJ) appear in one system and coordinate together for contraction. The gut spheroid-derived GIO of the present invention expresses SMC, ENS, and ICJ as required for a motile gut generation in the culture of the invention, which was only possible to generate from the unique gut spheroids of the invention. (Here, expresses means protein expression of intestinal specific tissue proteins or genes which verified or established or demonstrated intestinal and its associated cells.)

Thus, the GIO of the invention has highly convoluted tissues, including α-smooth muscle actin (SmoothA)/β-actin and uniaxonal/multipolar neurons, as reported in publications^{14, 15}, producing a contractile gut. Reported publications have tried to replicate such a system from in vivo mice or human intestinal isolated cells, which can't follow human developmental pathways similar to human stem cell-derived organoids.^{13, 14} The HiPSCs-derived motile gut of the invention coordinates 3D cytoarchitecture with SmoothA cells to produce contractile action, which is similarly observed in the human fetal intestine of the pseudovillus structure.¹⁵ The novel gut spheroids-derived human contractile GIO of the invention can be used to monitor GIO pathogen invasion and further GIO architecture changes can be determined using videos.

In one aspect of the invention, the GIO is a contractile, fully functional GIO that can maintain innate immune signaling pathways via secreting cytokines and chemokines in vivo. 16 The GIO of the invention was treated with bacterial lipopolysaccharide (LPS) in various reported physiological concentrations (10-100 fig/mL).^{17,18,19} It was found that 6 out of 10 different pro-inflammatory cytokines (ProC) such as interleukin (IL)-2, IL-6, IL-8, tumor necrosis factor (TNF)-α, macrophage inflammatory protein (MIP)-1α, and MIP-1β were upregulated with LPS (10-100 μg/mL) in a dose and time-dependent fashion (24-48 h time point). Nevertheless, only 3 ProC, such as IL-10, IL-1β, and interferon-gamma (IFNγ) were upregulated when treated with only 100 μg/mL LPS for 48 h since their signaling depends on the upregulation of IL-2, IL-15, and IL-18. 20,21 Moreover, IL-10, IL-1β, and IFNγ can abrogate endotoxin, thus need a higher dose to activate, similar to in vivo, as observed in the various studies. 20,21,22,23 Thus, the novel complex GIO of the invention mimics the in vivo cytokine regulatory pathway, which can be explored for studying gastroenteritis and related inflammation.

In another aspect, the invention relates to making a fully functional complete GIO from 3D gut spheroids. Usual intestinal organoids rise from stem cell-derived endodermal adherent cells, but the GIO of the invention is directly derived from 3D self-organized and renewal spheroids, which can directly be exposed to hind/midgut and intestinal growth factors in a stepwise manner. The 3D gut spheroids of the invention were exposed to Activin A (100 ng/mL), Wnt3A, FGF4 (50 ng/mL), EGF, and R-spondin (200 and 10 ng/mL) (see, e.g. de Lau, W. B., Snel, B. & Clevers, H. C. The R-spondin protein family. Genome Biol 13, 242 (2012). https://doi.org/10.1186/gb-2012-13-3-242.) Activin A is added to the spheroids on Day 3, and Wnt3A and FGF4 are added on Day 6, while on Day 11 addition of Rspondin is started. The inventors also utilized overall growth factor concentrations of 50-100 ng/mL and observed similar GIO morphology and functionality in these ranges. 3D gut spheroid invention produced the most convoluted microtissues that mimic human GIO as observed in the culture system.

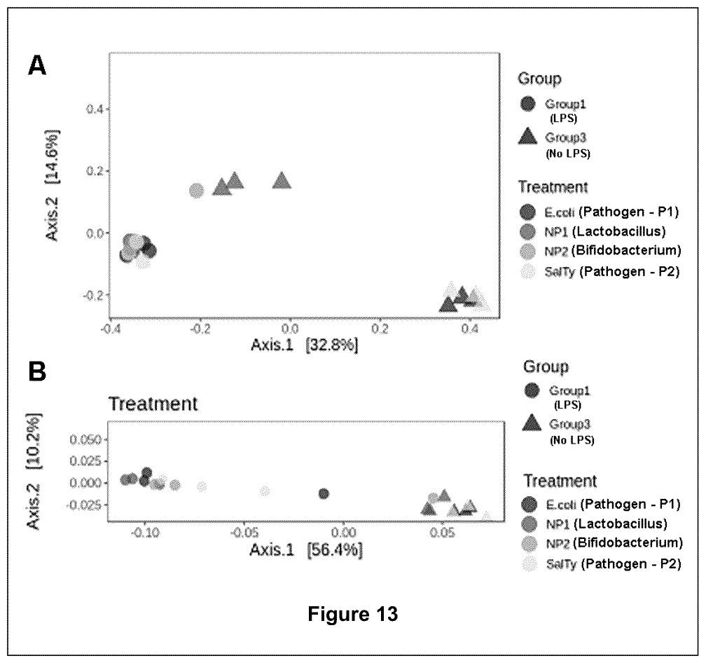

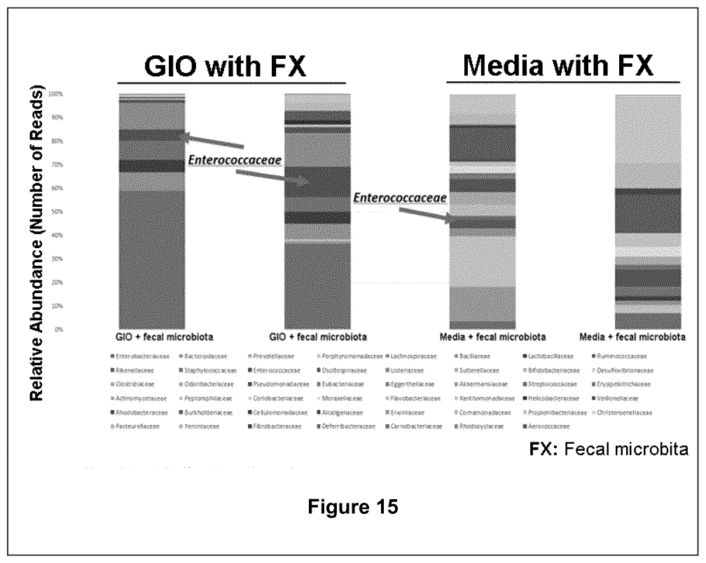

The invention also relates to host-microbe interaction using GIO through shotgun metagenomics analysis. The inventors developed a method where the inventors used pathogen and non-pathogenic bacteria to GIO and treated them with human fecal microbiota with or without LPS (Sus-GIO). For the addition of fecal microbiota, 100 mg of fecal microbiota was dissolved with sterile PBS and filtered by a 70 μm cell strainer to remove aggregates. Filtered fecal microbiota was further diluted 1:100 using an intestinal expansion medium without penicillin/streptomycin. After that, UT-GIO and Sus-GIO were treated with diluted fecal microbiota and pathogenic/non-pathogenic bacteria. An important part of the invention is that the GIO behaved like a complete human intestinal system with a metagenomic profile with proportional distribution of bacteria changes. No in vitro GIO system yet provides bacterial changes with LPS or pathogens with the microbiota further altered bacterial community. Our shotgun metagenomic invention takes place:
(i) LPS versus non-LPS conditions to understand our GIO can capture susceptibility to pathogen conditions, which causes a bloom of an unknown Bacteroides sp. (Bacteroidales bacterium M2), and the Bonferroni test showed significant abundance compared to GIO without LPS (*p<0.05). The GIO of the invention can hold the susceptibility to infection and interact with the microbiome.
(ii) Beta diversity (Unifrac distance) analysis color-coded using LPS. Each point is a metagenome annotated by the taxonomy of the bacteria present. The distance between the dots is a representation of the phylogenetic distance between the communities in each sample. Weighted means that the proportional abundance of each taxon has been included in calculating community distance, and unweighted means that proportional abundance has been excluded from that calculation. Weighted ‘weights’ abundant taxa, whereas unweighted ‘weights’ rare taxa. The % on the axes references the variation in the microbial community structure described by the first two (ranked) dimensions, as discussed in FIG. 13.
(iii) As part of the invention, the inventors treated the GIO samples with fecal microbiota versus untreated samples and found bacterial proportion significantly changed. The inventors also compared GIO versus media (non-GIO) with microbiota and found NGL GIO itself accelerates the abundance of the Enterococcaceae bacterial family known to balance Lactobacillus in GIO. These results are the first to verify that in vitro GIO can hold/regulate the microbiome system and successfully study disease, infections, and host-microbe interaction, as shown in FIG. 15.

3. Making the GIO of the invention.

The process for making the GIO of the invention generally comprises:
a) culturing HiPSCs in a first culture media to produce 3-D gut spheroid cells;
b) treating said gut spheroid cells with Activin A;
c) treating said gut spheroid cells with Wnt3A and fibroblast growth factor 4 to produce large gut spheroid cells having a diameter of greater than about 200 μm;
d) seeding said large gut spheroids on a dome-shaped gel-matrix in an intestinal media containing EGF, noggin, and Rspondin; and
e) culturing the resulting dome-shaped gel matrix comprising said gut spheroid cells to provide a complete gastrointestinal organoid.

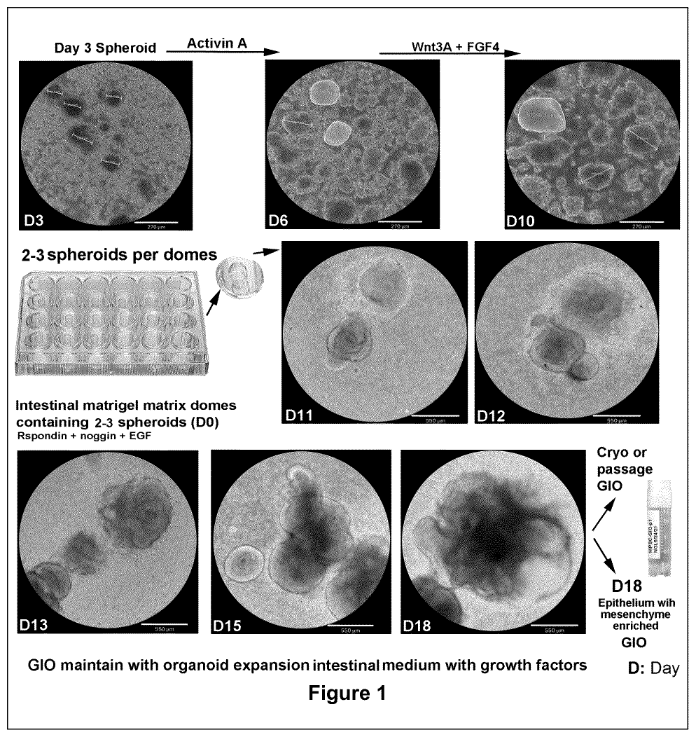

A time course of mature gastrointestinal organoids (GIO) development. As shown in FIG. 1, the method of the invention first generates gut spheroid from HiPSCs at 50-150 μm within the day Day(D)3-D10. On day 3, spheroids were treated with human recombinant Activin A (100 ng/mL). On Day 10, spheroids started to become big with bright edges and darker colors (>200 μm) by treating with 50-100 ng/mL of human recombinant Wnt Family Member 3A (Wnt3A) ((see, e.g., Hirokawa Y, Yip K H, Tan C W, Burgess A W. Colonic myofibroblast cell line stimulates colonoid formation. Am J Physiol Gastrointest Liver Physiol. 2014; 306(7):G547-56)) and fibroblast growth factor 4 (FGF4). Then gut spheroids are seeded with gel-matrix domes and maintained with GIO growth medium started to become >1 mm GIO at about Day 11-Day 12. The gel-matrix domes can be comprised of various biocompatible gels, such as polymeric gels, such as acylated chitosan mixed with a gel matrix, such as Matrigel and geltrex. The matrix gel-containing organoid fragments are pipetted into the center of a well in a 24-well tissue culture-treated plate to form a dome (20 μL-30 μL in a 24-well plate) which we called gel matrix domes. Gel matrix (Matrigel-Corning Inc and Geltrex Matrix—Gibco, both of which contain extracellular matrix protein, laminin, collagen, and others) are domes comprising a hollow upper half of a sphere where organoids grow. Gut spheroids started attaching and appearing paracellular barrier with GIO architecture, as shown in the D 12-13 images. Further, GIO started to become larger with microscopic visualized epithelial, lumen, and others. On day 18, GIO confluent >90% of domes, and this GIO is ready to passage. This GIO can be passage >10 times and expanded in culture or cryopreserved (Cryo), scale bar 550 μm. D; Day.

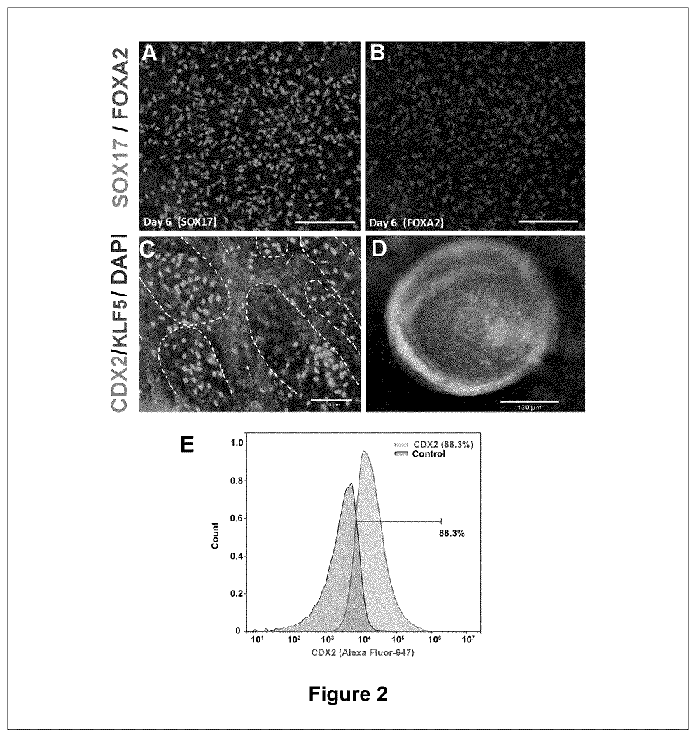

Morphogenesis of posterior endoderm and hindgut. As shown in FIG. 2, during endodermal differentiation, the cell appears to be a distinct flat and confluent sheet of definite endoderm (DE), as observed in fetal intestinal development in vivo. During the first ten days in culture, spheroids expanded into organoids, giving rise to a pseudostratified epithelium that co-expresses the fetal intestinal transcription factors (A) SOX17 (green) and (B) FOXA2 (red) expression, which verified successful endodermal differentiation. (C) CDX2 (green) hindgut, and (D) KLFS (red) lumen and crypt types specification. (E) Flow cytometry measurement verified that CDX2 cell (Alexa Fluor 647) expressed >85% (red arrow). Once CDX2 marker expression reaches >85% via flow cytometry measurement, gut spheroid seed into intestinal gel matrix domes to obtain complete GIO. GIO inside the intestinal matrix dome started to mature and express mature intestinal markers with complex tissue layers (dotted arrow) observed during fetal mouse intestinal development. Scale bar 70-130 μm.

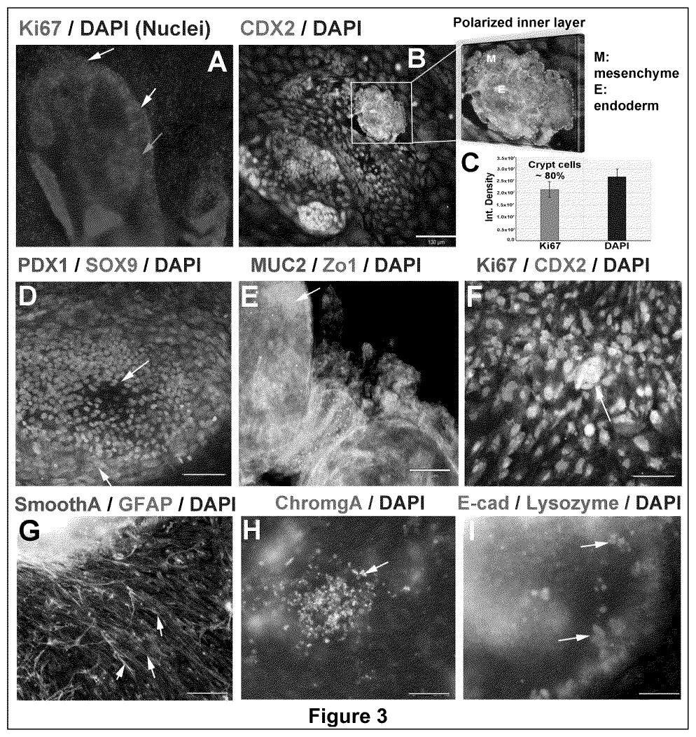

Formation of HiPSC-derived fully functional GIO. As shown in FIG. 3, the spheroid cells of the invention, when plated with gel matrix domes, generates a fully functional GIO by 18 days with the expression of (A) crypt-type proliferating cells (Ki67), (B) polarized inner endodermal and mesenchyme (CDX2⁺). (C) The inventors also quantified crypt-type proliferating cells via Ki67 marker cells which were determined with the comparison of nuclei (DAPI) in the same proliferating zone. The inventors found ˜80% of the cells represent crypt-type cells. (D-F) Mature GIO also expressed higher levels of endocrine cells (PDX1+) and SOX9 (stem cell proliferation+), goblet cells (MUC2+/MUC6+), intestinal crypts proliferating cells (Ki67+). (G-I) α-smooth muscle actin (smooth A) and glial fibrillary protein (GFAP) coordination led to generating contractile gut, which expressed in our GIO including enteroendocrine (ChromG, chromogranin A+) and highly convoluted epithelial E-cadherin (E-cad+) and Paneth cell-derived lysozyme. All these expressions indicating that the GIO of the invention is fully functional. Scale bar 130 μm or higher. Arrow indicates a specific marker expression.

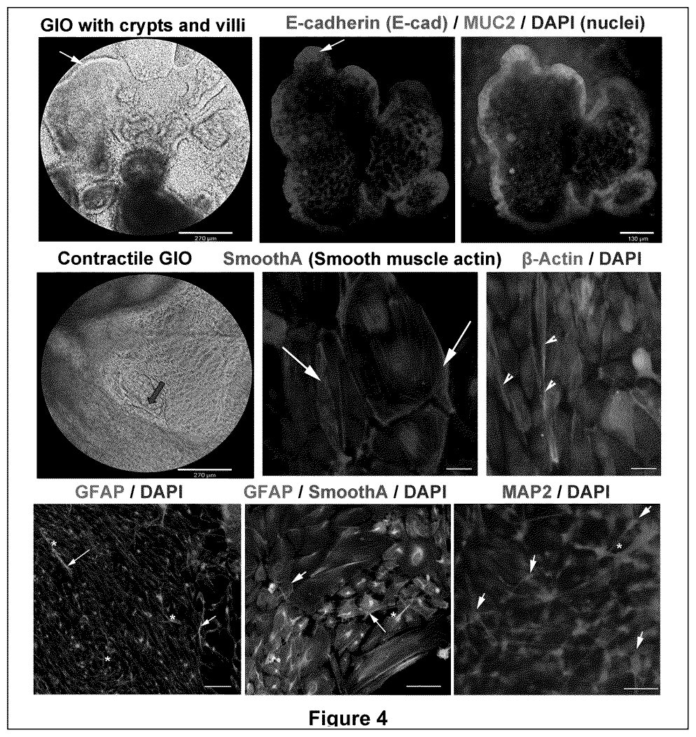

Formation of contractile GIO with marker expressions. As shown in FIG. 4, The inventors have generated (A) 3D GIO from HiPSC-derived gut spheroids consisting of a polarized columnar epithelium with villi and crypts patterning with the expression of (B-C) E-cad (red) and MUC2 (green). (D) Our GIO showed spontaneous contraction after day 28 with highly convoluted epithelial structures (E-cad, red) surrounded by mesenchyme, as reported in publications.^{11,14} (E-I) Contractile activities (red arrow) require preserving and coordinating α-smooth muscle actin (SmoothA, red), β-actin (green, white arrowhead), and uniaxonal/multipolar glial cells (GFAP, green), neurons (MAP2, red) coordinating with SmoothA (red) which the inventors observed here (arrow showed specific expression cells). Scale bar 130 μm or higher. White star (*) represented branch type GFAP cells or MAP2 neuronal soma.

### EXAMPLES

### Example 1

### Gut Spheroids Culture Protocol

Human-induced pluripotent stem cell (HiPSCs) were purchased from Creative Bioarray, Shirley, NY. HiPSCs were seeded 3×10⁶ cells per well of 6 well plates.

Step 1 (Spheroid Generation): Once cell confluence >90% cell is dissociated by gentle cell dissociation reagent (StemCell Technologies) followed by plating of dissociated fragmented cells in serum replacement base medium consisting of knockout serum replacement (Invitrogen), DMEM/F12 (Invitrogen), glutamax (Invitrogen), β-mercaptoethanol (Invitrogen), 10 μM of rock inhibitor, 10 ng/mL of BfGF and EGF and 5 μg/mL of heparin (Sigma-Aldrich). All the growth factors except heparin were purchased from StemCell Technologies.

Step 2 (Gut Spheroid Generation): The cultured cells were kept for 5 days until spheroids became large, over 100 μm, then the spheroids were treated with and cultured with endodermal differential medium (RPMI 1640, glutamax, pen/strep from Millipore Sigma) consisting of activin A growth factor (100 ng/mL, StemCell Technologies) for three days.

Step 3 (Mid/hindgut generation): At day 4, the spheroids are cultured with mid/hindgut differentiation medium consisting of RPMI 1640, glutamax, FGF4 (50 ng/mL), and wnt3A (50 ng/mL) for 7 days. After 10 days, the gut spheroid appeared highly translucent with double-edged epithelium with a size of over 200 μm. Gut spheroids at this point are ready to seed in gel-matrix domes for further differentiation to become fully functional intestinal organoids, namely the GIO of the invention.

### Example 2

### GIO In-Vitro Development Protocol

Step 4 (Gastrointestine generation): Gut spheroids larger than 200 μm in size are collected in a 1.5 ml Eppendorf tube and placed at 4° C. until put in gel-matrix domes. The gel matrix is a matrix combination consisting of 0.5% acylated chitosan in Matrigel or Geltrex matrix gel (available from Corning Inc. and Gibco). A variety of useful acylated chitosans are known and can be produced by the reaction of chitosan with a variety or organic acids and derivatives of organic acids, (mainly anhydrides and acyl chlorides), introducing aliphatic or aromatic acyl groups to the molecular chain. The inventors added 25 μl of cooled gel-matrix drop to 2-3 gut spheroids containing cool Eppendorf tube, gently picked these gel-matrix containing gut spheroids, and seeded them on 24 well plates. Once the gel becomes solid, media is added, the intestinal medium consisting of advanced DMEM/F12, B27 supplement (1×, Invitrogen), 15 mM HEPES buffer (MilliporeSigma), Rspondin (100 ng/mL, R&D Systems), Noggin (StemCell Technologies, 100 ng/mL) and EGF (200 ng/mL). Gut spheroid-containing gel-matrix domes were maintained with intestinal medium for 8 days, which led to fully functional mature GIO within 18 days, expressing restricted crypts (Ki67⁺), goblet cells (MUC2⁺), Paneth cells (lysozyme⁺), endocrine cells (PDX1⁺), and enteroendocrine cells (chromogranin A⁺) compared to the reported system require over 30 days 7,10,11,12 The GIO system of the invention is rapid and robust. This is the first reported method to derive mature GIO from gut spheroids with double-edged epithelium surrounded by stratified mesenchyme that contains smooth muscle cells, and sub-epithelial fibroblasts recapitulate complete and contractile human GIO in vivo.

### Example 3

### Contractile GIO Demonstration

Periodic contraction of intestinal muscles can only arise from the coordinated activity of smooth muscle cells (SMC), the enteric nervous system (ENS), and the interstitial cells of Cajal (ICJ), which is quite challenging to develop. In the GIO system of the invention, when it was cultured over 28 days with an intestinal medium, we observed large GIO >1 mm with smoothA, β actin, desmin, GFAP, and MAP2 marker cell expression substantially increased, which was determined by ICC. We also compared our gut spheroid-derived GIO with traditional adherent endodermal culture-derived intestinal organoids. We found traditional culture method-derived intestinal organoid has no contractile activity. Thus, NGL's unique novel in-house double-edge epithelial 3D gut spheroids with acylated chitosan-gel matrix were able to produce highly convoluted epithelium consisting of SMC, ENS, and ICJ and produce contractile gut, which resembles the mini-intestinal architecture of human in vivo.

### Example 4

### Fluorescent Imaging and Analysis

Immunocytochemistry (ICC) and flow cytometry is a common cellular protein or antigen-detecting and visualization techniques that can recognize the target of interest or specific marker expression in cells via imaging and a series of flow histograms. The antibody is directly or indirectly linked to a reporter, such as a fluorophore or an enzyme. We performed ICC and flow cytometry as a previously established method developed by the PI.^{35, 36} Briefly, for flow cytometry, cells were collected and dissociated with trypsin into a single cell and then blocked with 2% mouse serum and staining with conjugated anti-CDX2 antibody (BD Biosciences) as previously established method¹¹ and run to NovoCyte Flow Cytometer (Agilent Technologies, Santa Clara, CA). We have verified endodermal marker verification using CDX2 expression using flow cytometry. But for ICC, we have grown GIOs on coverslips coated or directly on the 24-well plates. Then cells were washed with PBS (phosphate-buffered saline) and fixed with 4% paraformaldehyde (PFA, Thermo Scientific). After additional washes in PBS, the cells were permeabilized in 0.1% Triton X-100 (Sigma-Aldrich), followed by blocking with 5% BSA (bovine serum albumin) in PBS containing 10% normal goat serum (NGS, Thermofisher Scientific). Next, the cells were incubated overnight with primary antibodies diluted in a blocking solution at CC with an appropriate dilution indicated by the manufacturer. The next day, the cells were washed three times with washing buffer (1×PBS containing 0.05% Tween 20 and 1% NGS). Then cells were incubated for 2 h at 25° C. with a fluorescent secondary antibody (Life Technologies). GIOs were further washed, and the coverslips with the cells were placed onto slides with a Fluoromount-G mounting medium containing 4′,6-diamidino-2-phenylindole (DAPI-FG, Southern Biotech), but GIO without coverslips were incubated with DAPI dye (Thermo Scientific). We performed ICC in various stages of GIO to determine their marker expression, including day 18, to observe DE specification, intestinal epithelium, polarized and nonpolarized intestinal cells, crypt-like cells, goblet, and Paneth cells. We verified the expression of MUC2 and MUC6 (goblet cell), PDX1 (enteroendocrine), lysozyme (Paneth cell), chromogranin A (endocrine cell), SOX9 (transit-amplifying zones of the crypt), Ki67 (restricted crypts like expression), Zo1 (tight junction protein) which occurred in the fetal mouse at E17. All antibodies were purchased from BD Biosciences or Invitrogen. NGL lab-produced GIO followed a similar developmental pathway in the fetus or fetal mouse³⁷, including smooth muscle contractile ability determined by smoothA and β-actin,¹⁴ GFAP, and MAP2.

### Example 5

### Cytokine Array Analysis

10-plex proinflammatory cytokine (ProC) measurement of LPS and pathogen/non-pathogenic bacterial treated GIO media (supernatant) was assayed using the Luminex multiplexing cytokine suspension array technology (Immune Monitoring Core Facility, USC, CA). Measurements of IL-2, IL-6, IL-8, IL-10, IL-13, TNF-α, INF-γ, MIP-1α and MIP-1β in the supernatant were analyzed using a MilliPlex MAP human cytokine/chemokine magnetic bead panel (MilliporeSigma, St. Louis, MO). Plates were read on a Bio-Plex 200 System using Bio-Plex Manager software version 6.0 (BioRad, Hercules, CA). Five (5) parameter logistic regression algorithms were used to determine cytokine concentrations based on provided cytokine standards and averages from triplicate-wells. Each ProC standard was measured using six known concentrations (10.0 ng, 2.0 ng, 0.4 ng, 0.08 ng, 0.016 ng, 0.0032 ng), and most regression values reached >0.99. Limit of detection: The lower limit of quantitation (LLOQ) for each ProCs are IL-13 (0.257 pg/mL), IL-1β (0.485 pg/mL), IL-2 (0.157 pg/mL), IL-8 (0.299 pg/mL), TNF-α (0.382 pg/mL), IL-6 (0.179 pg/mL), INF-γ (0.562 pg/mL), IL-10 (1.510 pg/mL), MIP-1α (0.273 pg/mL), MIP-1β (881.9 pg/mL). We quantified the UT-GIO, LPS-treated Sus-GIO, and Cryo-GIO with or without pathogenic and non-pathogenic bacteria. After analyzing ProC in each treatment, we conducted statistical analyses to determine the significance of the ProC status of pathogenic versus non-pathogenic bacteria in LPS-treated (Sus-GIO) and untreated GIO (UT-GIO) using GraphPad Prism (San Diego, CA). We have performed two-way ANOVA with Tukey's multiple comparisons using GraphPad Prism. *p<0.05, **p<0.01, ***p<0.001, ****p<0.0001 denotes significance.

### Example 6

### In-Vitro Lucifer Yellow Dye Diffusion Assay

Lucifer Yellow (LuciY) dye (Invitrogen) was added in 1 mM concentration to GIO initially treated with LPS and pathogenic bacteria to assess intestinal paracellular barrier permeability. The concentration of dye that diffused through the membrane into endothelial/epithelium to GIO core 28 was analyzed by ImageJ, and fluorescence intensity (FI) was calculated using the following formula:

$$GIOin = \frac{( Vin - Vout )}{Vout} \times 100$$

LuciY dye FI was measured at 485/535 nm using an Echo fluorescence Revolve microscope (Echo, San Diego, CA). The amount of LuciY dye inside the GIO was determined using the formula where Vin is the intensity of the dye entered inside the GIO core. Vout is LuciY dye intensity measured outside or in the background of the organoids. GIO intracellular (in) FI was calculated by quantifying each image's integrated density. Using this equation, we quantified the dye permeability inside the GIO core due to the infection and damage or leaking to the intestinal membrane. FI is higher in GIO means greater intestinal epithelium damage occurred via specific treatment conditions, represented in the graph by integrated density. GraphPad Prism was used to determine the significant difference between pathogenic versus non-pathogenic bacterial-induced intestinal leaking using a one-way ANOVA with Tukey multiple comparisons tests (p<0.05).

### Example 7

### Microbiota Enables GIO System Developed

Our UT-GIO and Sus-GIO were treated with fecal microbiota to generate GIO with a complete gut-microbiome system observed in vivo.^{38,39} In this context, we first prepared fecal microbiota, purchased from Discovery Life Sciences (DLS). DLS obtains fecal microbiota samples according to their protocol approved by their internal review board (IRB). Then, 100 mg of fecal microbiota was dissolved with sterile PBS and filtered by a 70 μm cell strainer to remove aggregates. Filtered fecal microbiota was further diluted 1:100 using an intestinal expansion medium without penicillin/streptomycin. After that, UT-GIO and Sus-GIO were treated with diluted fecal microbiota and pathogenic/non-pathogenic bacteria to examine whether fecal microbiota altered or rescued pathogen-mediated intestinal barrier cell destruction and microbiome profiles using shotgun metagenomics analysis. Microbiota-treated GIO with or without LPS and pathogenic/nonpathogenic bacterial culture media sample was analyzed by University of California, San Diego (UCSD) Microbiome Core to obtain metagenomic profiles.

### Example 8

### Shotgun Metagenomics

In total, pathogenic and non-pathogenic UT (untreated) and Sus (susceptible)-GIO samples were processed for total DNA extraction and metagenomic library preparation and sequencing. Sample plating and extractions of all samples were conducted in a biosafety cabinet Class II in a BSL2⁺ facility of Microbiome Core of UCSD. Samples were plated into a bead plate from the 96 MagMAX™ Microbiome Ultra Nucleic Acid Isolation Kit (A42357 Thermo Fisher Scientific, USA). Following the KatharoSeq low biomass protocol, each sample processing plate included eight positive controls consisting of 10-fold serial dilutions of the ZymoBIOMICS™ Microbial Community Standard (D6300 Zymo, USA) from 5 to 50 million cells per extraction. Each plate contained at least 8 negative controls (sample-free lysis buffer). Nucleic acid purification was performed on the KingFisher Flex™ robots (Thermo Fisher Scientific, USA) using the MagMAX™ Microbiome Ultra Nucleic Acid Isolation Kit (Applied Biosystems™), as instructed by the manufacturer. Briefly, 800 μL of lysis buffer was added to each well on the sample processing plate and centrifuged to bring all beads to the bottom of the plate. Sample swab heads were added to the lysis buffer and firmly sealed with MicroAmp™ transparent adhesive film (Thermo Fisher Scientific, UK) using a sealed roller. The sealing process was repeated twice using foil seals. The plate was beaten in a TissueLyser II (Qiagen, Germany) at 30 Hz for 2 min and subsequently centrifuged at 3700×g for 5 min. Lysates (450 μL/well) were transferred into a Deep Well Plate (96-well plate, Thermo Fisher Scientific, USA) containing 520 μL of MagMax™ (Applied Biosystems, Thermofisher Scientific) binding bead solution and transferred to the KingFisher Flex™ for nucleic acid purification using the MagMax™ protocol. Nucleic acids were eluted in 100 μL nuclease-free water and used for downstream preparation. Gene amplification was performed in 384-well plates with unique Illumina forward and reversed primers with a single reaction per sample. Equal volumes of each amplicon were pooled, and the library was sequenced on the Illumina NovaSeq platform. All QA and QC steps were completed and validated, providing high confidence in the data product quality. Sequencing data were processed first by performing adapter trimming and human-read filtering via pairwise alignments using minimap2. All reads were uploaded into qiita, and taxonomy was assigned using the Web of Life toolkit (WoLtka) implementation in Qiime2 via QIITA (read more here: https://github.com/qiyunzhu/woltka). Beta diversity analyses were performed in R using the package phyloseq, using Weighted and Unweighted UniFrac as a primary metric. Proportional taxonomic analyses were estimated for taxonomic features at family and species levels.

### Example 9

### In-Vitro MTT Cell Viability Assay

We determined GIO viability after the freeze-thaw cycle using a colorimetric assay to evaluate cell metabolic activity with an MTT [3-(4,5-dimethylthiazol-2-yl)-2,5-diphenyltetrazolium bromide] assay, following the manufacturer's instructions for the assay (MilliporeSigma). To perform the reported established MTT assay⁴⁰, we grew GIO in 24-well plates and then treated them with or without LPS for 96 h to observe viability and stability. We added lmg/mL of MTT reagent to each −well containing GIO, followed by the 2 h incubation at 37° C. Further, GIO was washed with sterile PBS, followed by the addition of dimethyl sulfoxide (DMSO) in each—well and mixed. After the 10 min incubation, DMSO was absorbed the MTT and run for a plate reader (SpectraMax iD3 Plate Reader, Molecular Devices, San Jose, CA) with the optical absorption density (OD) of 570 nm to determine the amount of MTT binding in the viable cell. This method determined our GIO's viability and stability after the freeze-thaw cycle with or without inflectional susceptible LPS treatment. We calculated the amount of MTT in each well using the following formula:

$$Cell Viability ( \% ) = \frac{OD 570 treated GIO}{OD 570 control GIO} \times 100$$

GraphPad Prism was used for comparing significant differences between treatments using an unpaired t-test (p<0.05).

4. Evaluating the GIO of the invention.

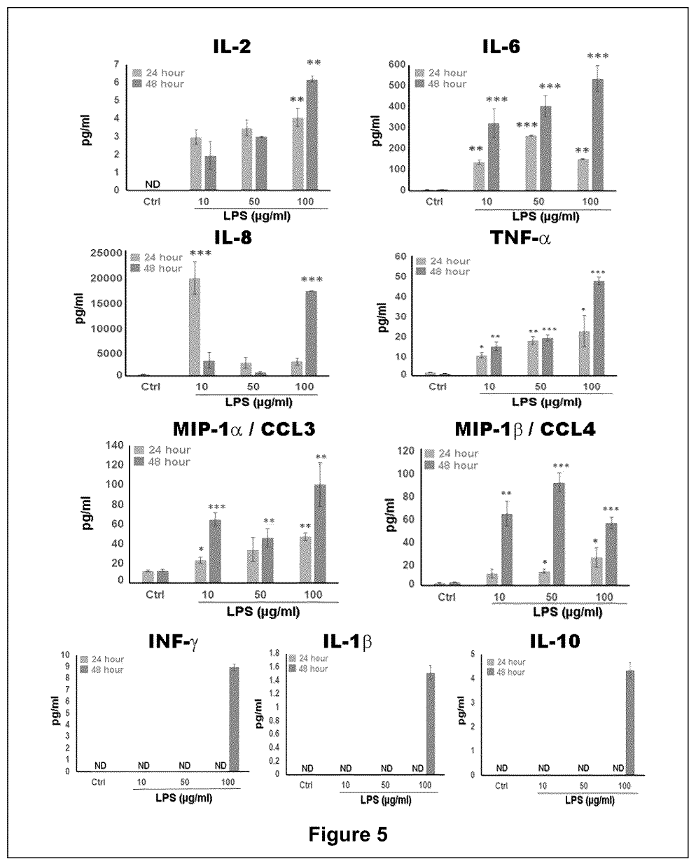

Susceptible (Sus) GIO was developed to study pathogen infection. As shown in FIG. 5, GIO was treated with bacterial endotoxin LPS with different concentrations to understand our GIO can recapitulate microbe-host interactions similar to humans in vivo. The inventors found 6 out of 10 different ProC and chemokines such as IL-2, IL-6, IL-8, TNF-α, MIP-1α, and MIP-1β were upregulated with LPS (10-100 μg/ml) in a dose and time-dependent fashion [24 h (blue bar) and 48 h (purple bar) time point] using 10-plex cytokine array, but IL-10, IL-1β, and IFNγ were upregulated only in 48 h time points with 100 μg/mL LPS dose. IL-13 was unable to detect with this time point or any concentration of LPS. GraphPad Prism was used for comparing significant differences between treatments using an unpaired t-test. p<0.05, **p<0.01, ***p<0.001, ****p<0.0001 denotes significance. Star (*) compared control vs. LPS.

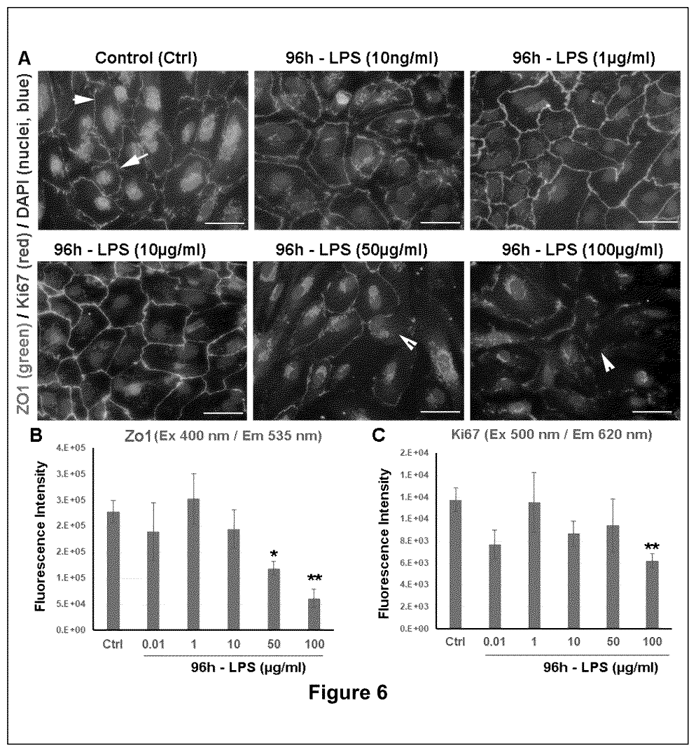

Effect of chronic LPS induction on TJP1 (tight junction protein 1). (A) As shown in FIG. 6, tight junctions are protein complexes that create intercellular boundaries between the plasma membrane domains of epithelial and endothelial cells. One of the critical TJP is the Zo1 protein located on a cytoplasmic membrane surface of tight intercellular junctions that can be used for the marker of intestinal damage or dysfunction. Our study observed that the LPS (50-100 μg/ml) affects GIO TJP1 at 96 h. The inventors also performed Zo1 immunostaining at an earlier point (24-48 h) but could not see any effect on Zo1. Ki67, an intestinal crypt cell type, decreased by LPS 100 μg/ml. (B-C) Zo1 and Ki67 quantification of fluorescence intensity (FI) showed 50-100 μg/ml LPS effect Zo1 and Ki67 significantly at 96 h time. Scale bar 70 μm. The inventors have performed one-way ANOVA with Tukey's multiple comparisons using GraphPad Prism. *p<0.05, **p<0.01, ***p<0.001, ****p<0.0001 denotes significance. Star (*) compared control vs. LPS.

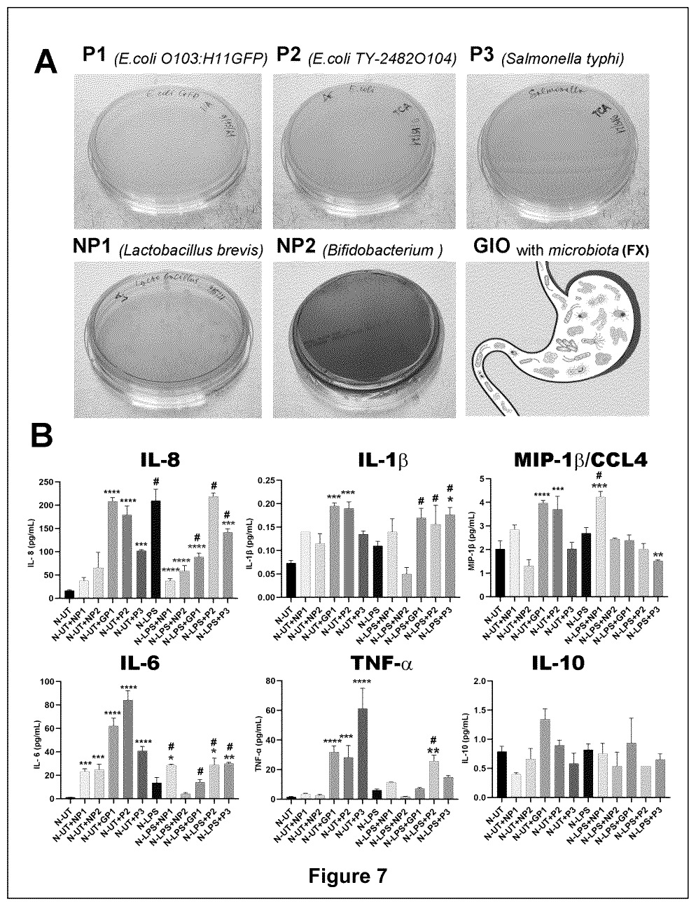

Pathogen versus non-pathogenic bacterial response with 10-plea cytokine array. (A) As shown in FIG. 7, To study the pathogen versus non-pathogenic effect on GIO, the inventors have used three strains of pathogenic bacteria (Escherichia coli strains: TY-24820104:H4 and O103:H11GFP and Salmonella Enterica serovar Typhi) and two strains of non-pathogenic bacteria (Lactobacillus Brevis; Bb14, Bifidobacterium adolescentis) from ATCC (Manassas, Virginia). These bacteria were cultured using TSB (Tryptic Soy Agar/Broth), NB (Nutrient Agar/Broth), and LB (Lactobacilli MRS Agar/Broth). The inventors maintained a pure and stable bacterial culture, producing over 1×10⁷ bacterial cells/mL within 24 h. (B) UT-GIO and LPS-induced infection Sus-GIO was treated with three pathogens and two non-pathogens. The inventors have found IL-6, IL-6, IL-8, TNF-α, and MIP-1β were upregulated with pathogen with 8 h treatment without LPS significantly, but LPS (100 μg/ml) treatment with pathogens caused a reduction to the ProC. Some of the ProCs were also undetected or lower, such as IL-10, IL-1β, IFNγ, and MIP-1α. The inventors have observed a pathogen versus non-pathogen effect on proinflammatory cytokine production. The inventors have performed two-way ANOVA with Tukey's multiple comparisons using GraphPad Prism. *p<0.05, **p<0.01, ***p<0.001, ****p<0.0001 denotes significance. Star (*) compared control vs. pathogens but (*) pound comparison pathogen vs. non-pathogen. N (normal or Fresh GIO), UT (untreated), GP1 (E. coli-GFP), P2 (E. coli second pathogenic strain), P3 (S. typhi), NP1 (Lac, L. brevis), NP2 (Bf, B. adolescents), FX (fecal microbiota).

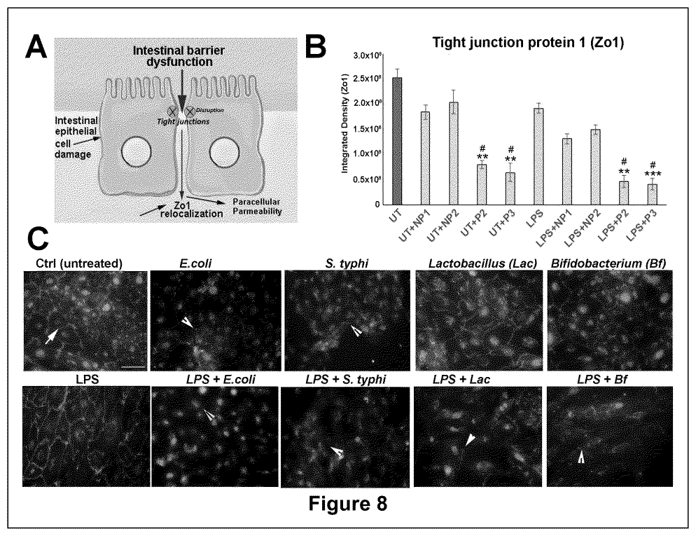

Intestinal tight intracellular junction disruption by the pathogen. (A) As shown in FIG. 8, Intestinal tight junction disruption by pathogens led to the reduction of TJP1 marker cells Zo1. As part of the novelty, the inventors want to determine if the GIO of the invention with pathogens can recreate humans' in vivo system. (B) The inventors quantified Zo1 marker cells in our GIO after exposure to pathogens using ImageJ software. The inventors have performed two-way ANOVA with Tukey's multiple comparisons using GraphPad Prism. *p<0.05, **p<0.01, ***p<0.001, ****p<0.0001 denotes significance. Star (*) compared control vs. pathogens but (*) pound comparison pathogen vs. non-pathogen. Lac (Lactobacillus, L. brevis), Bf (B. adolescentis). (C) GIO cells were immunostained with anti-Zo1 and Ki67 antibodies, and it was found that pathogen-treated cells significantly reduced Zo1 (green)/Ki67 (red) expression even though there had nuclear staining (DAPI, blue). Interestingly nucleus was floating, and pathogens completely disrupted cytoplasm walls. The inventors have observed that non-pathogenic bacteria have a small effect on Zo1 of LPS-induced GIO. Sus-GIO might be sensitive, and non-pathogenic bacteria might play an opportunistic role. Scale bar 130 μm or higher. The yellow arrow showed Zo1 staining, as observed with a long white arrow. Arrowhead indicated reduced Zo1 staining or small nuclei. Yellow arrowhead fragmented nuclei.

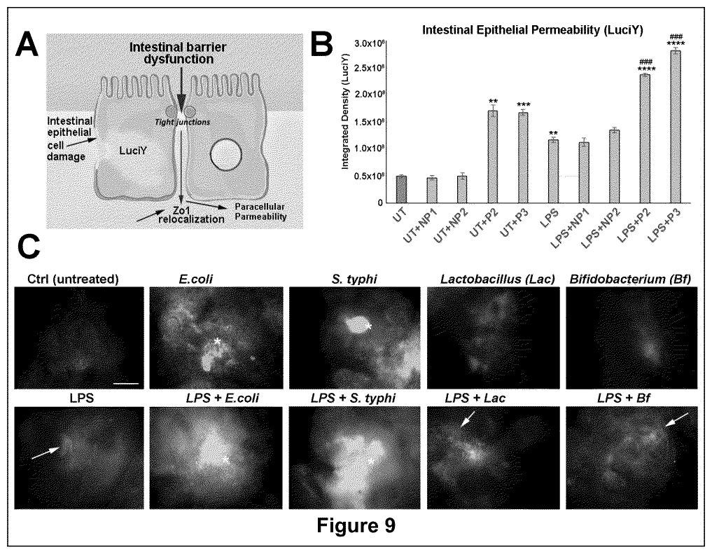

Intestinal barrier defects induce paracellular permeability and leaking by the pathogen. (A) As shown in FIG. 9, pathogen damage to intestinal epithelial cells led to a dysfunctional intestinal barrier and leaked the paracellular barrier, which usually happens in vivo. As part of the novelty, the inventors want to determine if pathogens in our GIO can recreate humans in vivo. (B) The inventors have quantified the pathogen effect on GIO paracellular damage and leaking by analyzing LucY dye internalization using ImageJ software (NIH). The inventors have performed one-way ANOVA with Tukey's multiple comparisons using GraphPad Prism. *p<0.05, **p<0.01, ***p<0.001, ****p<0.0001 denotes significance. Star (*) compared control vs. pathogens but (*) pound comparison pathogen vs. non-pathogen. (C) Fluorescence microscopic observation showed pathogens treated GIO absorbed a significant amount of LucY (lucifer yellow) dye compared to non-pathogen-treated GIO or UT-GIO. Moreover, the pathogen-treated Sus-GIO core has an excessive amount of LuciY dye compared to other treatments. Thus, infection-susceptible conditions accelerate GI infection, which can permanently damage gastrointestine (GI) and may not be reversible. Arrow indicated leaking of intestinal epithelial. Lac: (L. brevis, Lactobacillus), Bf: Bifidobacterium strain, and E. coli and S. typhi as pathogenic bacteria as the inventors used in the experiment. Lac (L. brevis, Lactobacillus), Bf (Bf, B. adolescentis). White star (*) showed LucY dye in the GIO core, and the white arrow showed the entrance of LucY dye: scale bar 130 μm or higher.

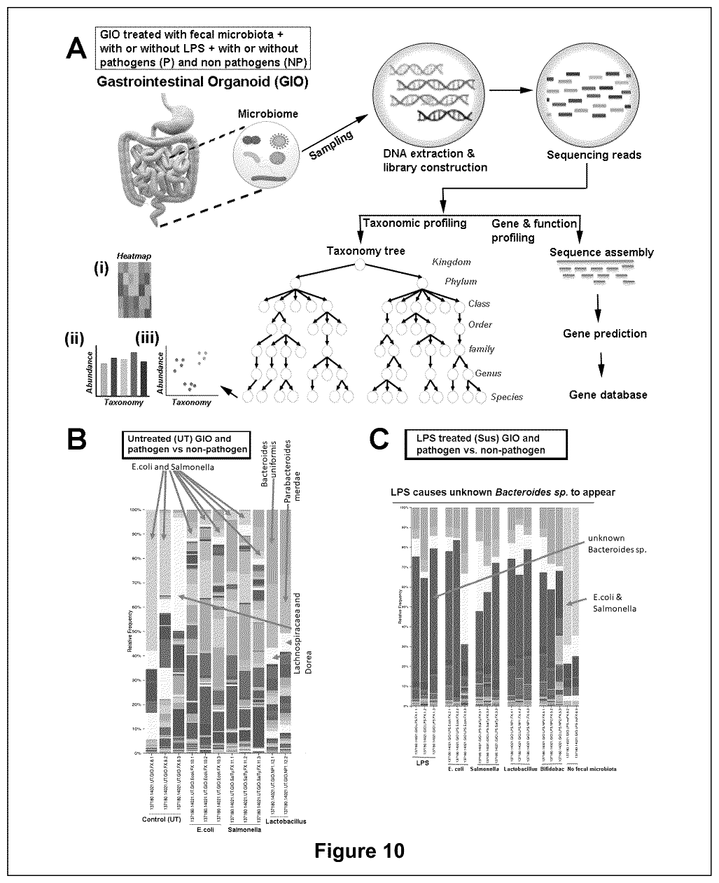

Microbiota enables a complete GIO system developed as human in vivo. (A) As shown in FIG. 10, GIO was treated with fecal microbiota to determine whether our GIO can recapitulate the human in vivo microbiome, which adds novelty to our method. Stepwise schematic of shotgun metagenomics for our GIO where the inventors collected samples after the treatment of GIO, extracted genomic DNA, and ran for metagenomics. Beta diversity analyses were performed in R using the package phyloseq, using Weighted and Unweighted UniFrac as a primary metric. Proportional taxonomic analyses were estimated for taxonomic features at family and species levels. The taxonomic data were quantified and represented according to treatment as shown in (i) heatmap, (ii) bar graph, and (iii) clusters. Microbiota GIO treated with pathogen and non-pathogen to determine the metagenomics profile of bacteria. Representative heatmap of (B) UT-GIO and (C) LPS-treated GIO. LPS causes the unknown species' appearance (Bacteroides sp.) in GIO culture, which is absent from UT.

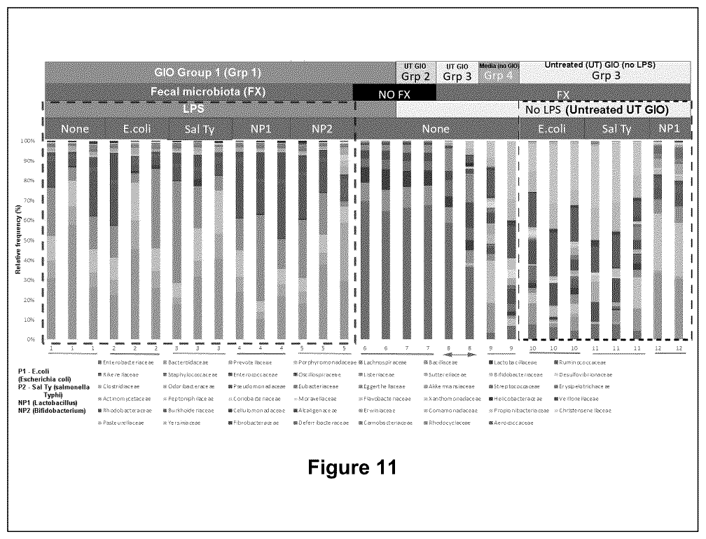

Shotgun metagenomics to determine the proportional distribution of bacteria changes in the heatmap. As shown in FIG. 11, a representative heatmap of shotgun metagenomics. The color-coded bacterial changes are shown in this heatmap. This map illustrates LPS versus (vs) non-LPS GIO (Untreated—UT) causes significant changes in bacterial proportion in each group. The dotted square represents LPS versus non-LPS (untreated) group changes bacterial family represented by a color-coded bar with pathogenic and non-pathogenic bacteria treatment. Fx (fecal microbiota), P1 (E. coli), P2 (Salmonella Typhi), NP1 (Lactobacillus brevis), NP2 (Bifidobacterium adolescentis). Group 1 (LPS), Group 2 (no Fx and no LPS), Group 4 (Media with Fx), and Subgroup 3 with a double arrow (UT-GIO) with Fx.

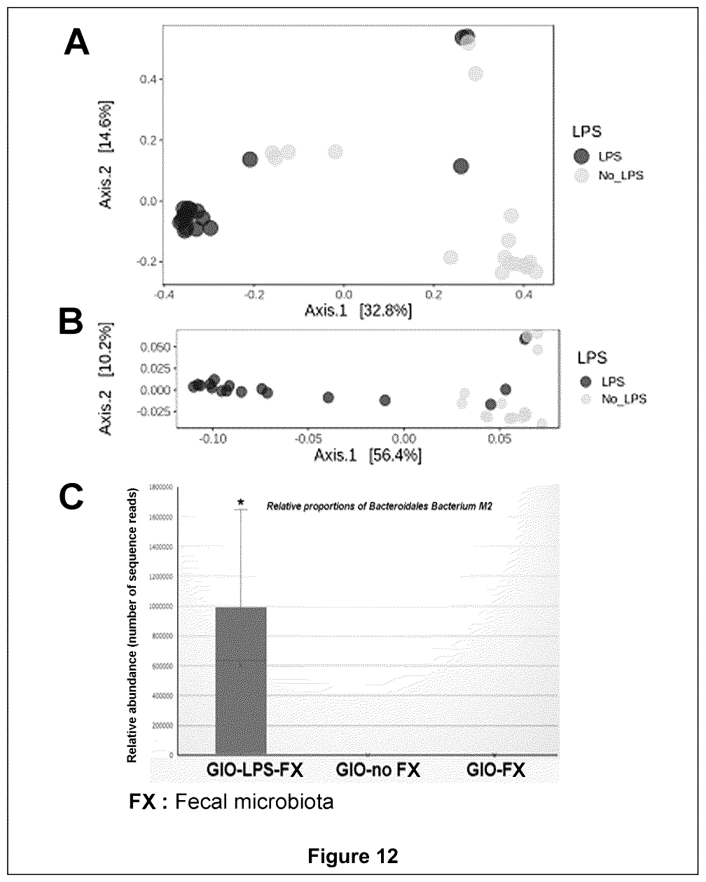

The cluster graph of LPS versus non-LPS showed significant alteration of the bacterial community. (A) As shown in FIG. 12, Unweighted Unifrac; (B) Weighted Unifrac. Each point is a metagenome annotated by the taxonomy of the bacteria present. The distance between the dots is a representation of the phylogenetic distance between the communities in each sample. Weighted ‘weights’ abundant taxa, whereas unweighted ‘weights’ rare taxa. The % on the axes references the variation in the microbial community structure described by the first two (ranked) dimensions. (C) LPS causes a bloom of an unknown Bacteroides sp. (Bacteroidales bacterium M2), and the Bonferroni test showed significant abundance compared to GIO without LPS (*p<0.05). Fm; Fecal microbiota, GIO; gastrointestinal organoid.

Beta diversity (Unifrac distance) analysis color-coded using various groups with treatment conditions. (A) As shown in FIG. 13, Unweighted Unifrac; (B) Weighted Unifrac. Each point is a metagenome annotated by the taxonomy of the bacteria present; the distance between the dots represents the phylogenetic distance between the communities in each sample. Weighted means that the proportional abundance of each taxon has been included in calculating community distance, and unweighted means that proportional abundance has been excluded from that calculation. Weighted ‘weights’ abundant taxa, whereas unweighted ‘weights’ rare taxa. The % on the axes references the amount of variation in the microbial community structure described by the first two (ranked) dimensions.

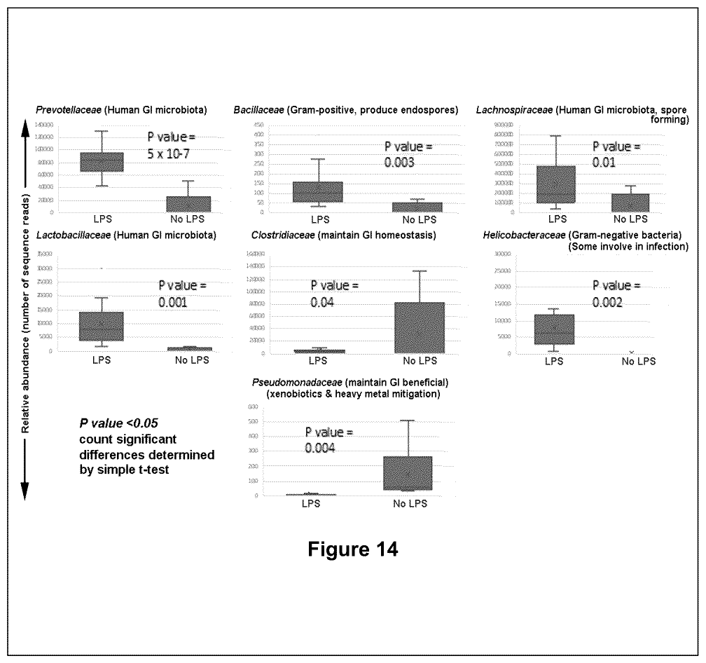

Dominant bacterial families change with LPS. As shown in FIG. 14, box and whisker plots demonstrate the median, upper, and lower quartiles and outliers for the relative proportion of dominant bacterial families, including the significant p-value (<0.05) between Group 1 and Group 3 taxa. The x-axis shows Group 1 and Group 3; the y-axis is the proportional abundance in reads annotated to each taxon. The bar graph represents a bacterial family that caused a major change in the microbiome.

GIO can hold in vivo human microbiomes with microbiota. As shown in FIG. 15, the novelty of the GIO of the invention is in keeping and recreating the microbiome system with fecal microbiota in GIO is similar to in vivo human intestinal system. The inventors have compared the relative abundance of specific bacterial families between untreated organoids with a fecal microbiome (GIO+fecal microbiota) and organoid media with a microbiome (Media+fecal microbiota), the Enterococcaceae bacterial family that is significantly differentially abundant (p<0.003). The Enterococcaceae are a family of Gram-positive bacteria placed in the order Lactobacillales. Representative genera include Enterococcus, Melissococcus, Pilibacter, Tetragenococcus, and Vagococcus. Some essential lactic acid bacteria in this family produce lactic acid as the major metabolic product.

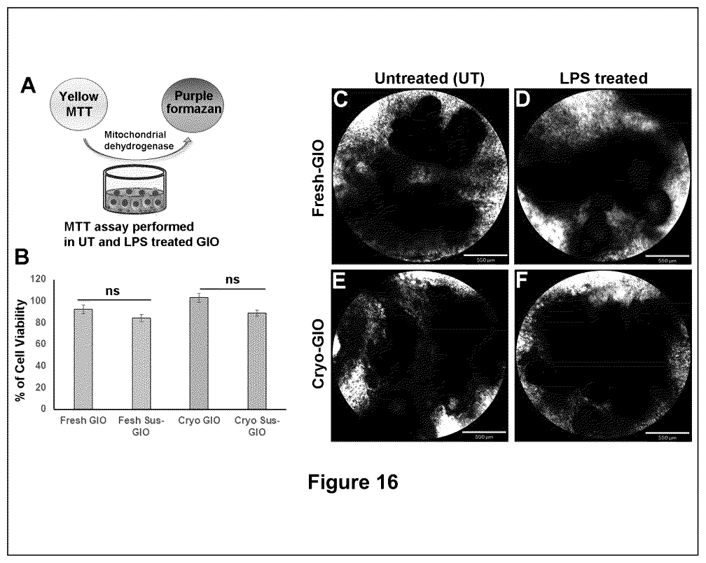

Fresh versus Cryo-GIO viability and stability. (A) As shown in FIG. 16, the MTT [3-(4,5-dimethylthiazol-2-yl)-2,5-diphenyltetrazoliumbromide)] assay is used to measure cellular metabolic activity as an indicator of cell viability, proliferation, and cytotoxicity. (B) To perform the MTT assay, the inventors grew GIO in 24 well plates and then treated GIO with or without LPS for 96 h to observe GIO viability and stability. LPS lost cell viability <15-20%, a minimal and non-significant difference. (C-E) Viable cells with an active metabolism converted MTT (yellow color) into a purple-colored formazan product and were measured with a microplate reader with an absorbance near 570 nm. Thus, our cryopreserve GIO (Cryo-GIO) is highly viable after the freeze-thaw cycle and can be used extensively for clinical or research use. UT: Untreated; LPS: Lipopolysaccharide.

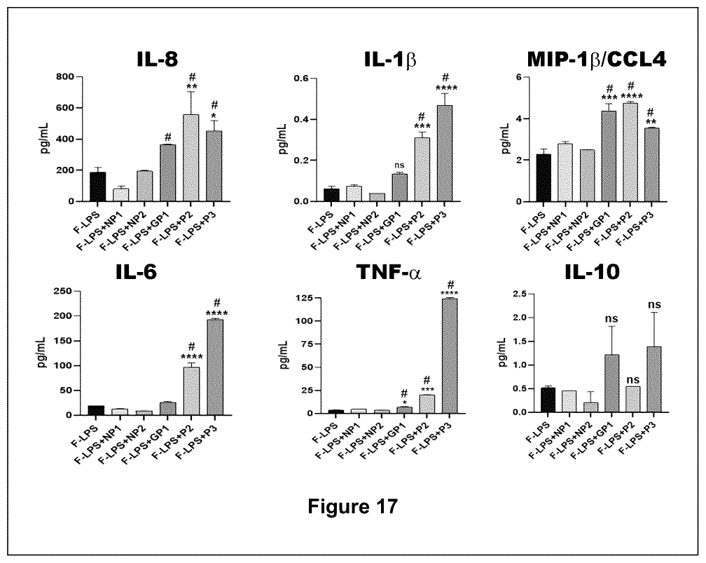

Fresh versus Cryo-GIO stability to secrete cytokines after pathogen infection: As shown in FIG. 17, a 10-plex cytokine array in Cryo-GIO was performed where Cryo-GIO was treated with LPS further was added pathogenic (P1, P2, P3), and non-pathogenic (NP1, NP2) bacteria to observe the upregulation of ProC productions using a 10-plex cytokine array (USC, CA). ProC results demonstrated that IL-8, IL-113, MIP-113, IL-6, TNF-α, IL-10 upregulation by pathogens significantly. Thus, Cryo-GIO has the capability to secret cytokines similar to fresh GIO, which can be easily commercialized and used in any facility to screen pathogen, drug, and their toxicity. The inventors have performed one-way ANOVA with Tukey's multiple comparisons using GraphPad Prism. *p<0.05, **p<0.01, ***p<0.001, ****p<0.0001 denotes significance. Star (*) compared control vs. pathogens but (*) pound comparison pathogen vs. non-pathogen. F (Frozen or Cryo-GIO), GP1 (E. coli-GFP), P2 (E. coli second pathogenic strain), P3 (S. typhi), NP1 (L. brevis), NP2 (B. adolescentis).

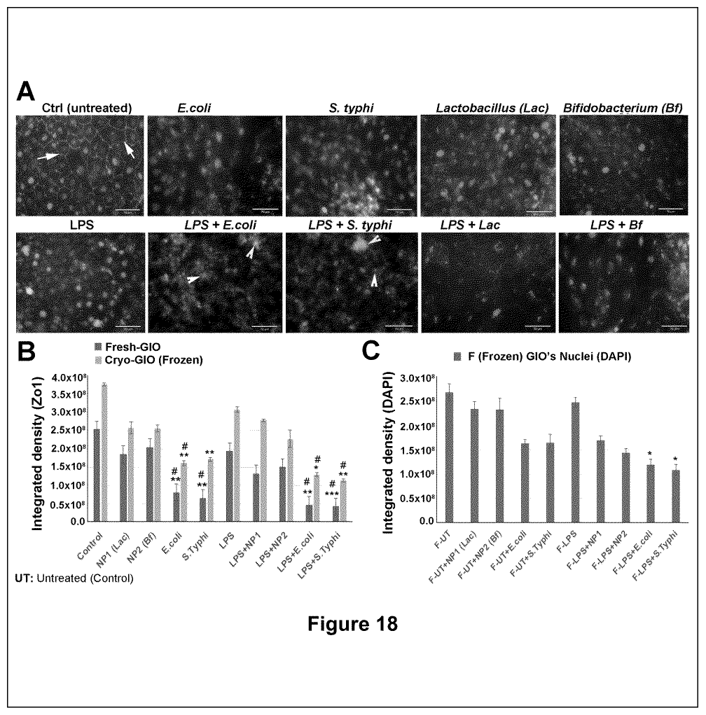

Fresh versus Cryo-GIO on TJP stability with or without pathogens: As shown in FIG. 18, Cryo-GIOs were treated with pathogenic and non-pathogenic bacteria further immunostained with anti-Zo1 (green) and -Ki67 (red) antibodies. (A) The inventors found that pathogen-treated cells significantly reduced Zo1 (green) and Ki67 (red) expression even though there has some nuclear staining (DAPI, blue). Interestingly GIO epithelium with Zo1 was aggregated and was completely disrupted by pathogens, as shown in the yellow arrowhead. The long white arrow represents the Zo1 expression in UT control. Zo1 (white arrow) is still visible in non-pathogenic bacteria treated with GIO. Lac: L. brevis, Bf: Bifidobacterium (B. adolescentis). LPS-induced GIO with pathogen showed small nuclei (white arrowhead) Scale bar 70 μm. (B) The inventors also quantified Zo1 marker cells in our Cryo-GIO after exposure to pathogens using ImageJ software. The inventors have performed two-way ANOVA with Tukey's multiple comparisons using GraphPad Prism. *p<0.05, **p<0.01, ***p<0.001, ****p<0.0001 denotes significance. Star (*) compared control vs. pathogens but (*) pound comparison pathogenic vs. non-pathogenic bacteria. (C) Nuclei were only significantly reduced in the LPS with pathogenic bacteria treated GIO sample, but samples without pathogenic bacteria didn't damage any nuclei significantly. Therefore, our GIO is highly stable and viable after cryopreserve and qualified for commercial use for pathogen or toxicity screening.

GIO quality was determined to verify its novelty use in high throughput study. In this context, the inventors have determined the viability and stability using MTT [3-(4,5-dimethylthiazol-2-yl)-2,5-diphenyltetrazoliumbromide)] assay. MTT assay is used to measure cellular metabolic activity as an indicator of cell viability, proliferation, and cytotoxicity that confirmed our Cryo-GIO stability is similar to Fresh-GIO. Using the guidance, these systems may be toxic if cell viability goes 70%. But our Fresh-GIO and Cryo-GIO overall maintained cell viability 80%. The inventors also verified Cryo-GIO stability to validate that our novel GIO in vitro can be stored in LN2 and used indefinitely for screening pathogens, drugs, and toxicity as follows:
(i) After evaluating the stability of Cryo-GIO with LPS treatment, the inventors treated Cryo-GIO with LPS (100 μg/ml) for 24 h. Further, the inventors treated them with pathogenic and non-pathogenic bacteria for 8 h, followed by the collecting media, and ran for determining 10 ProC by Luminex Multiplexing Cytokine Array (USC, CA). The inventors found that Cryo-GIO had successfully differentiated pathogenic from non-pathogenic bacteria, similar to the Fresh-GIO sample. The inventors also found that 6 out of 10 ProC was upregulated, including IL-6, IL-8, IL-1β TNF-α, and MIP-1β. However, the IL-2, IL-10, IL-13, IFNγ, and MIP-1α ProC detection was lower or undetected like Fresh-GIO, which confirmed our Cry-GIO stability and broader use for the experiment.
(ii) The inventors also found that Cryo-GIO with LPS and pathogens was able to damage GIO as similar to Fresh-GIO with a significant reduction of Zo1 marker expression compared to non-pathogens. Cryo-GIO response on Zo1 protein reduction by pathogens was also similar to Fresh-GIO. The quantification of Zo1 and nuclei (DAPI) using ImageJ also demonstrated that Cryo-GIO could differentiate pathogenic versus non-pathogenic bacteria like the Fresh-GIO with or without LPS treatment. Thus, our Cryo-GIO can be used for HT-scale pathogenic detection or toxicity screening due to its high stability as Fresh-GIO.

5. Using the GIO of the invention.

One aspect of the present invention is the development of a tissue complexity human cellular model that can rapidly determine pathogen or drug toxicity as an in vivo human physiological system compatible with HT testing. To do this, the present inventors generated highly functional HiPSC-derived GIO, which has tissue complexity and motile behavior as the human GI tract. This GIO has all the functional tissues or gland type-cells for secreting and transporting hormones or enzymes with anti-microbial activity. Fecal microbiota is introduced in the GIO that behaves and maintains the microbial communities as the human gut. The GIO of the invention can differentiate pathogenic versus nonpathogenic bacteria even with susceptible conditions such as human GIO developed by LPS, verified by determining ProC secretion, intestinal paracellular barrier leaking, TJP1 expression, and shotgun metagenomics.

The present invention utilizes an electroconductive system, such as one by the Applied Biophysics Inc. team using their Electric Cell-Substrate Impedance Sensing (ECIS) base. With electric cell-substrate impedance sensing (ECIS) technology (such as Applied Biophysics, Inc.) where the impedance change of a current flow through the cell culture medium in an array plate (ECIS-based electroconductive plate) is measured in a non-invasive manner. ECIS® (Electric Cell-substrate Impedance Sensing) is a real-time, label-free, impedance-based method to study cell behaviors in tissue culture. The cell behaviors that can be studied include monolayer barrier function, cell growth rates and viability, wound-healing migration, and other behaviors directed by the cell's cytoskeleton. The ECIS® approach has been applied to numerous investigations, including measurements of endothelial monolayer permeability, in vitro toxicity testing as an alternative to animal testing, the invasive nature of cancer cells, and signal transduction involving GPCRs for modern drug discovery. ECIS® uses a small non-invasive alternating current (I) that is applied across the electrode pattern at the bottom of the ECIS arrays (direct current cannot be used). This results in a potential (V) across the electrodes which is measured by the ECIS instrument. The impedance (Z) is determined by Ohm's law Z=V/I. When cells are added to the ECIS Arrays and attached to the electrodes, they act as insulators increasing the impedance. As cells grow and cover the electrodes, the current is impeded in a manner related to the number of cells covering the electrode, the morphology of the cells, and the nature of the cell attachment. When cells are stimulated to change their function, the accompanying changes in cell morphology alter the impedance. The data generated is impedance versus time. (See Applied BioPhysics, https://www.biophysics.com/whatIsECIS.php) ECIS has been used to study cellular responses to (a) pathogen/virus infection (See, Pennington et al, Electric Cell-Substrate Impedance Sensing To Monitor Viral Growth and Study Cellular Responses to Infection with Alphaherpesviruses in Real Time, ASM Journals, mSphere, Vol. 2, No. 2, 5 Apr. 2017); (b) environmental factors (See, Amalu Navas, Maya Nandkumar A. Electric Cell-Substrate Impedance Sensing (ECIS) for Analyzing the Effect of Environmental Pollutants—A Study of Diesel Exhaust Nanoparticles. Journal of Environmental Science and Public Health 6 (2022): 189-203; (c) drug/therapeutic evaluation (see Adcock, A. F., Agbai, C. O. & Yang, L. Application of electric cell-substrate impedance sensing toward personalized anti-cancer therapeutic selection. J Anal Sci Technol 9, 17 (2018); drug toxicity screening (see, Tran et al, Hydrogel-based diffusion chip with Electric Cell-substrate Impedance Sensing (ECIS) integration for cell viability assay and drug toxicity screening; Biosensors & Bioelectronics, 17 Jul. 2013, 50:453-459); and other aspects of activities and morphologies of cells (see, Zhang X, Jang S. The application of electric cell-substrate impedance sensing (ECIS) biosensors. Int J Biosen Bioelectron. 2018; 4 (6):26010.15406/iibsbe.2018.04.00136.

Further, the complex GIO is grown on a multiwell electroconductive plate, such as an HT-scale 96-well arrays. The inventors compared a pathogen-treated GIO signal and compared with an electrical signal. The electroconductive system of the invention has two-electrodes inserted in each well with a smaller electrode diameter and connected with an insulating electrode. The electric cell-substrate impedance sensing (ECIS) culture system consists of sterile disposable electrode arrays containing gold film electrodes delineated with an insulating film.

GIO was grown in the 96-well electrode sensor arrays, which signal was highly sensitive compared to the 8-well arrays. Also, the inventors examined GIO electrical signals with or without pathogenic and non-pathogenic bacteria. The electrical signal from the 96-well arrays with pathogenic bacteria was highly specific and comparable to untreated or non-pathogenic GIO.

The GIO of the invention with an electroconductive plate prototype can produce highly sensitive signals from 96-well arrays with a 354 μm electrode. Thus, the fully functional GIO system of the invention with a multicellular organoid is constructed onto ECIS multichannel electrode plate. The 96-well arrays can be extended to 384-well for rapidly detecting harmful pathogen responses from an environment with a rapid and robust scale that can protect travelers and the general population from infection immediately. The novel, fully functional complex, contractile, and complete GIO system of the invention can be used for drug screening, environmental toxins, and underlying disease mechanisms of human intestines and replace animal studies. The system of the invention can screen over 50-300 pathogens, toxins, or drugs within 3-5 h in this human model system, depending on the HT plate format (96 or 384-well), but the same number of animal screenings will take 3 to 12 months. This ready-to-use human GIO system in HT sensing arrays is highly innovative, which the current market unable to offer for rapid and sensitive screening platforms like ours.

In vitro and in vivo studies linked the down-regulation of the scaffolding protein Zo1, encoded by the TJP1 (tight junction protein 1) gene, to determine tight junction permeability. 24,25,26 In this context, we have determined that 100 μg/mL of LPS within 96 h time point disrupts endothelial tight junction protein (TJP) via assessing the expression of anti-Zo1 (TJP1 expression)—antibody using immunocytochemistry (ICC). Using the innovative GIO of the invention, the present inventors generated an efficient susceptible (Sus)-GIO model verifying ProC secretion and TJP1 to study pathogen response.

5.1 Pathogen Screening

To examine pathogen screening using the GIO system of the invention, we cultured two pathogenic [Escherichia coli strains: O103:H11GFP (P1), TY-24820104 (P2): H4 and Salmonella enterica serovar Typhimurium (P3)] and two [Lactobacillus brevis; Bb14 (NP1), Bifidobacterium adolescentis (NP2)] nonpathogenic bacteria purchased from ATCC (Manassas, VA). Three pathogenic bacterial strains (E. coli, two strains, and S. typhi) and two nonpathogenic bacteria (L. Brevis and B. adolescentis) were used to treat the GIO of the invention. These bacteria were cultured in TSB (Tryptic Soy Agar/Broth), NB (Nutrient Agar/Broth), and LB (Lactobacilli MRS Agar/Broth). These bacterial cultures were collected from the broth. The number of bacteria (typically 1×10⁶ bacteria for each 24-well plate for 8-16 hrs) was determined before applying them to GIO for determining pathogenic versus non-pathogenic bacterial responses. The GIO 3D architecture with endothelial and epithelial was damaged substantially with pathogenic bacterial exposure via destroying the intestinal luminal barrier, further invasion of bacteria internally significantly.

Untreated (UT)-GIO and Sus-GIO (untreated and susceptible GIO) of the invention were further treated with pathogenic (E. coli and S. typhi) and nonpathogenic (L. brevis and B. adolescentis) bacteria to study innate immune response via secretion of cytokines using 10-plex cytokine arrays. Most of the ProC was upregulated (IL-6, IL-8, IL-10, TNF-α, and MIP-1V in UT-GIO with all pathogens. However, Sus-GIO had less response than UT-GIO on secreting IL-6, IL-8, IL-10, TNF-α, and MIP-113. Sus-GIO might damage excessively by pathogens to induce any prosurvival signal to further the production of ProC at higher levels. 27 IL-2, IL-13, MIP-1α, and IFNγ had no response regardless of UT or Sus-GIO condition. Overall, ProC secretion of pathogenic and non-pathogenic bacterial responses significantly differed regardless of UT-GIO and Sus-GIO.

The present inventors also observed that intestinal barrier permeability was compromised with pathogenic bacteria, which was observed through the Lucifer yellow (LuciY) dye entrance into the GIO core via diffusion of the damaged intestinal barrier. Furthermore, LuciY dye was observed in the GIO core, which demonstrated considerable damage to the intestinal epithelium by pathogens and diffusion of dye entrance into the GIO core. Nevertheless, non-pathogenic bacteria caused minimum damage to the intestinal barrier, and a low amount of LuciY dye entered the GIO. In addition, untreated GIO showed no leaking of the intestinal barrier cells with no visualization of LuciY dye in the GIO core. Thus, the present inventors were able to differentiate the pathogenic versus non-pathogenic bacterial response by intestinal permeability assay using LuciY dye as observed in various ex vivo and in vivo studies.^{28,29,30} Thus, in one aspect of the invention, the GIO responds to pathogens and non-pathogens usually observed in human in vivo systems so that the GIO of the invention can be used to study environmental pathogens.

As part of the invention, the present inventors examined the pathogenic and non-pathogenic treated UT-GIO and Sus-GIO to determine epithelial and endothelial TJP disruption via anti-Zo1 antibody using ICC. It was found that pathogenic bacteria caused considerable damage to intestinal epithelium via a reduction of Zo1 expression significantly. However, non-pathogenic bacteria had a negligible effect on the amount of reducing Zo1 protein regardless of UT-GIO or Sus-GIO. The inventors also determined that crypt-type proliferating cells by anti-Ki67 antibody staining to determine if pathogens affect overall intestinal morphology in the same GIO sample. It was found that pathogenic and non-pathogenic bacteria significantly decreased Ki67 expression like the Zo1 protein. ImageJ quantification (National Institute of Health Science, NIH) also verified that pathogenic bacteria significantly reduced Zo1 and Ki67 expression. The inventors also quantified nuclear staining by 4′ 6-diamidino-2-phenylindole dihydrochloride (DAPI) to confirm that cellular levels in each GIO were the same. The inventors observed a pathogen-mediated substantial response in our GIO with Zo1 and Ki67 staining similar to the human physiological response in vivo.

As part of investigating the innovative functionality of the GIO of the invention, the inventors treated the GIO with fecal microbiota with pathogens to obtain shotgun metagenomic profiles on UT-GIO and Sus-GIO. Shotgun metagenomic sample preparation and analysis were done by the University of California, San Diego (UCSD) of Microbiome Core. Proportional taxonomic features from the gDNA sample of treated GIO at the family and species levels were determined. 31 Beta diversity analyses were performed in R using the package phyloseq, Weighted and Unweighted UniFrac as a primary metric. 32 The taxonomic data were quantified and represented according to treatment using heatmap, bar, and cluster graphs. LPS-treated GIO caused a bloom of unknown Bacteroides sp. (Bacteriodales bacterium M2), which is not observed in the UT-treated GIO (p<0.0004). E. coli and S. typhi with UT-GIO significantly altered bacterial profiles compared to LPS-treated GIO. In addition, the inventors compared Sus-GIO versus UT-GIO on seven bacterial families and found the beneficial bacterial families significantly and proportionally increased in UT-GIO samples. But the infection-producing bacterial families (Helicobacteraceae) were decreased in UT-GIO. The inventors compared GIO versus media (non-GIO) with microbiota and found that the GIO of the invention accelerates the abundance of the Enterococcaceae bacterial family known to balance Lactobacillus in GIO. Thus, the GIO of the invention enables the improvement intestinal environment via increasing beneficial bacteria. Our GIO is one of the tremendous in vitro intestinal models of humans that can recapitulate gut-microbe interaction. Overall shotgun metagenomics result of beta diversity analysis (cluster color-coded graph) showed Sus-GIO versus UT-GIO group has significantly represented the phylogenetic distance between the bacterial communities in each sample. The difference between LPS versus non-LPS was significant by t-test, and the pathogen caused more changes to these differential responses. This kind of metagenomic profiles clarified that the GIO of the invention maintains the microbiome system and can have response with bacterial toxin like LPS and alter metagenomic profiles usually observed in human in vivo. Thus the GIO system of the invention allows the study of metagenomics status of GIO and the study of microbiome interaction with pathogenic species.

To determine viability and stability of the GIO of the invention, the present inventors tested cryopreserved GIO (Cryo-GIO) with fresh culture GIO by determining cell viability and stability by immunostaining of Zo1 and Ki67 marker proteins. We performed an MTT assay [3-(4,5-dimethylthiazol-2-yl)-2,5-diphenyl tetrazolium bromide)] of fresh versus cryopreserved (Cryo)-GIO; cell viability profiles were the same in both conditions. Both GIOs were treated with LPS for 96 h, and cell viability loss trends were similar for both cells (˜20%). Thus, our GIO is highly viable and stable after the freeze-thaw cycle. We also treated our Cryo-GIO with pathogenic and non-pathogenic bacteria as Fresh-GIO and stained for anti-Zo1 and -Ki67 antibodies. We found similar trends of reducing Zo1/Ki67 expression when treated with pathogenic bacteria. We also determined LPS and bacteria-treated GIO ProC secretion capability using a 10-plex cytokine array. Our data shows Cryo-GIO can significantly differentiate pathogenic versus non-pathogenic bacteria-mediate ProC signals (IL-6, IL-8, IL-113 TNF-α, and MIP-113) as Fresh-GIO. Thus, our Cryo-GIO is highly stable, can be stored and reused indefinitely, and can be marketed for researcher use.

As part of the invention, the complex GIO was grown on an electroconductive plate, which combined with the GIO system of the invention. The present inventors are the first to produce a GIO with high throughput electroconductive plates to rapidly determine environmental pathogens. In one embodiment, the present invention provides an electrode-based 8-well plate (8-well arrays) to grow GIO and optimize the GIO electric signal. (Other multi-well arrays can be used, including expanded 384 well plates or others.) As a result, the present inventors accurately established the GIO current/resistance compared to only media or epithelial monolayer cells. The inventors further developed the 96-well prototype, like the 96-well arrays of the Electric Cell-substrate Impedance Sensing (ECIS) system, to determine GIO compatibility with the high throughput electroconductive system. This prototype had a bigger electrode (354 μm) and inserted two electrodes in each well without an insulating electrode, which led to obtaining a more sensitive and robust GIO signal. The non-pathogenic bacterial electrical signal was like UT-GIO. Nevertheless, the pathogenic bacteria-mediated electrical signal was significantly different from UT-GIO or non-pathogens, which validates that the pathogenic bacterial signal has higher sensitivity and specificity. In addition, the pathogenic electrical signal represents the intestinal paracellular contact damage, which leads the current to flow easily with the production of low resistance. This response is similar to the reported physiological response in vivo.^{33, 34, 30}

The inventors also used 96-well arrays to determine Cryo-GIO and their electric signal with or without pathogenic and non-pathogenic bacteria. Cryo-GIO has a remarkably similar current/resistance signal to Fresh-GIO with pathogenic bacteria. Thus, the Cryo-GIO of the invention can differentiate pathogenic versus non-pathogenic bacterial signals after the freeze and thaw cycle as sensitive as the Fresh-GIO sample, so that the GIO of the invention combined with an electroconductive plate can be ready to use in pharmaceutical research by companies, research institutions, and academia for testing a drug, toxicity, environmental pathogens, and underlying disease mechanisms.

The inventors also assessed the signal-to-noise levels of cell culture media, GIO, and monolayer epithelium. The inventors found a unique and robust GIO electric signal of ˜2 nF compared to medium or monolayer epithelium ˜4-5 nF. So, the inventors were able to determine the accurate signal-to-noise levels of the GIO with or without treatment conditions, which is ≤0.5 nF or ≥4.2 nF (<1250 Ohms). The inventors assessed the electric signal of the GIO from 5 different frequencies, showed similar nF or Ohms levels, and differentiated pathogen versus non-pathogenic signal with 90% accuracy.

The inventors also compared our paracellular electrical signal once impedance breakdown by pathogens means resistance down significantly similar to the inventors observed with our pathogen-treated Zo1 expression in GIO. The electroconductive plate of the invention combined with optimized GIO current flow highly correlates (R²=0.86) with the Zo1 paracellular TJP1 protein, which is reduced after the pathogen invasion in the GIO. Thus, the GIO-grown electrode plate of the invention can recapitulate physiological response with pathogen invasion similar to humans in vivo.

The inventors evaluated the GIO capability to test pathogenic response using assays similar to reported physiological responses, such as 10-plex cytokines, shotgun metagenomics, LuciY diffusion assay, fluorescent Imaging for GIO protein markers, flow cytometry for endodermal marker expression, and electroconductive arrays. The inventors also explored markers (ProC, TJP1, DAPI, Ki67, LuciY dye) to verify pathogenic versus non-pathogenic bacterial responses in UT-GIO versus Sus-GIO. Microbiota enables a complete GIO system that can alter the composition of the microbial community. Pathogens or LPS changed bacterial communities and led new species to the microbial family in the GIO tissues of the invention. Thus, the GIO of the invention can produce a physiological response that resembles that of a natural human GIO. Furthermore, the electrode-derived GIO of the invention can produce highly distinguishable pathogenic signals comparable to non-pathogenic bacterial signals. The electric signal of the innovative GIO of the invention can differentiate any pathogenic bacterial response and rapidly and robustly detect pathogens in the environmental sample rapidly and robustly.

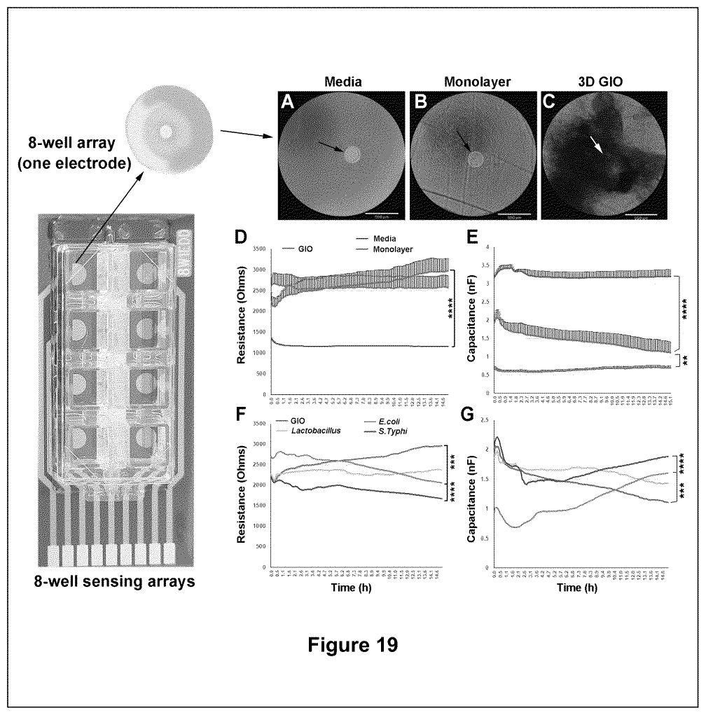

High throughput (HT) electroconductive sensing array prototype development for GIO signaling: As shown in FIG. 19, the inventors first designed and developed 8 well electrode arrays with ECIS base by fee-based service of Applied Physics, Inc. ECIS base grew Fresh-GIO in an 8-well array containing one electrode further determined (A) GIO electrical signal, which was significantly different than (B) monolayer epithelium and (C) media. The inventors plotted both (D-F) the resistance (Ohms) and (E-G) nanofarads capacitance (nF) signal curve at 64000 Hz. The inventors found pathogenic versus nonpathogenic bacterial response compared to GIO, which was highly significant (p<0.0001). Untreated (UT) GIO current was measured for 15 h using multifrequency. The inventors have performed two-way ANOVA with Tukey's multiple comparisons using GraphPad Prism. *p<0.05, **p<0.01, ***p<0.001, ****p<0.0001 denotes significance. Star (*) compared to control (GIO) vs. pathogens. The black and white arrow shows electrodes.

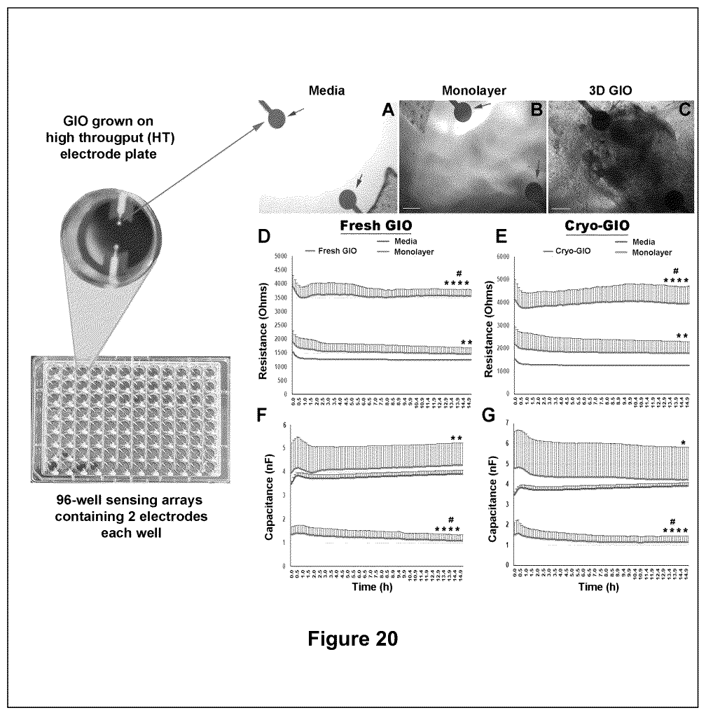

High throughput (HT) electroconductive sensing array development for rapid screening of fresh versus Cryo-GIO: As shown in FIG. 20, fresh vs. Cryo-GIO in 96 well electroconductive plates developed with ECIS base of Applied Biophysics, Inc (Troy, NY). The inventors have grown our Fresh-GIO and Cryo-GIO in HT 96-well electrodes containing two electrodes. The inventors further determined (A) GIO electrical signal, which was significantly different than (B) monolayer epithelium and (C) media. The inventors plotted both (D-E) the resistance (Ohms) and (F-G) nanofarads capacitance (nF) signal curve at 64000 Hz. Fresh and Cry-GIO signal was almost identical in 15 h run. The inventors have performed two-way ANOVA with Tukey's multiple comparisons using GraphPad Prism. *p<0.05, **p<0.01, ***p<0.001, ****p<0.0001 denotes significance. Star (*) compared to control (GIO) vs. monolayer but (*) pound comparison control vs. media. The red arrow shows electrodes. Our results demonstrated that optimized Cryo-GIO with an electroconductive plate could be ready-to-use without batch-to-batch variation to screen pathogens or toxicity as the first reported novel HT screening system.

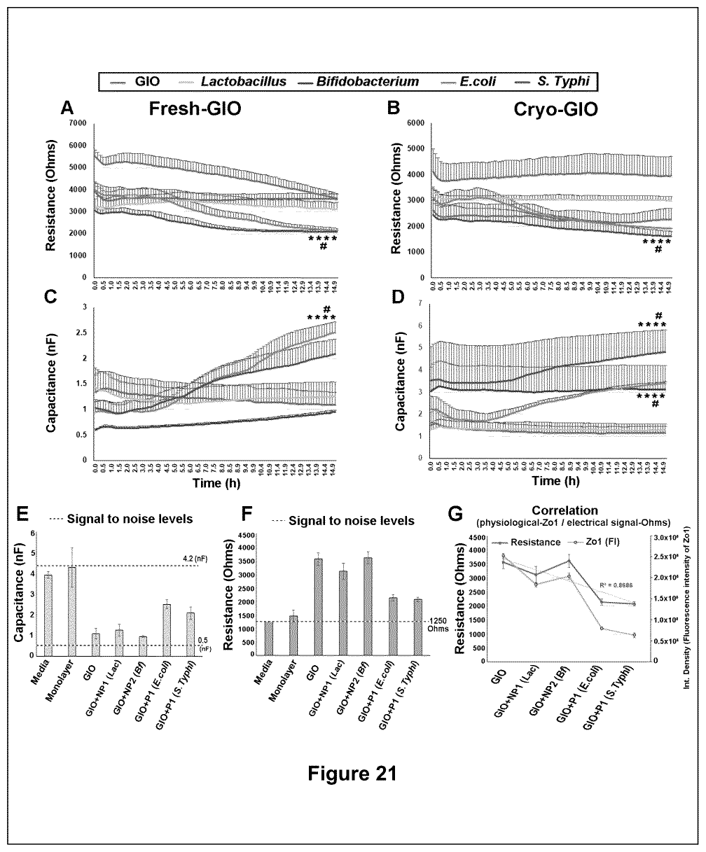

Correlation of HT electroconductive signal with tight junction protein of GIO: As shown in FIG. 21, fresh versus Cryo-GIO's capability to differentiate electrical signal from pathogenic versus non-pathogenic bacteria. The inventors have grown our (A, C) Fresh-GIO and (B, D) Cryo-GIO in 96-well electrodes, further determining treated and non-treated GIO's electrical signal. The inventors plotted both (A-B) the resistance (Ohms) and (C-D) nanofarads capacitance (nF) signal curve at 64000 Hz and determined pathogen versus non-pathogenic bacterial response compared to GIO (p<0.0001). The inventors have performed two-way ANOVA with Tukey's multiple comparisons using GraphPad Prism for the significant difference. *p<0.05, **p<0.01, ***p<0.001, ****p<0.0001 denotes significance. Star (*) compared to control (GIO) vs. pathogens but (If) pound comparison pathogenic vs. non-pathogenic bacteria. (E-F) Treated and non-treated GIO current was measured for 15 h using multifrequency to determine a signal to the noise level and stability to the electric signal over time. The dotted line below is noise for the resistance, but the dotted line below 0.5 nF or above 4.2 nF is the noise level for capacitance. (G) The correlation of electroconductive GIO combined plate electric signal with the paracellular contact junction protein (Zo1) levels validates cellular physiological response compatible with electric signals. Zo1 and Ohms signals were highly correlated (negative correlation) by performing linear regression (R²=0.86). TJP (Zo1) signal was reduced due to the damage of the GIO epithelium, which ultimately caused the resistance to decrease and showed a negative correlation. ECIS system, when cells decrease by toxicity on the electrode, reduces the resistance level of an electrical signal due to lower cell amount with the rapid electrical flow, which is exactly observed here. Thus the biological signal of Zo1 validates our novel electrical signal reliability to use our innovative GIO for laboratories, Big Pharma, and clinics.

In some embodiments, pathogens and bacterial strains such as E. coli, S. typhi, L. brevis, and B. adolescents culture in TSB, NB, and LB plate, but not limited this culture to this agar that can be possible to culture by other rich media blood supplemented agar plates. Our pathogenic bacteria (E. coli and S. typhi) and non-pathogenic bacteria (L. brevis and B. adolescents) were used in the culture with or without LPS. Various other combinations, such as pathogen and non-pathogen combinations with fecal microbiota or alone, can be used for learning host-microbe interaction in GIO. Pathogens with drugs, beneficial bacteria, or wide other varieties can be used to screen host-microbe interaction, drug efficacy, and toxicity against intestinal inflammation or diseases.

HT electroconductive sensing array combined with our optimized GIO is the first innovative technology that can be marketed for ready to use for screening pathogens, drugs, and toxicity at a rapid and robust scale.
(i) This invention generated GIO >1 mm, keeping the shape range from 2 to 5 mm in size with intestinal morphogenesis such as crypts, villas, brush borders, and lumen-type structures. The epithelium is surrounded by a stratified mesenchyme which contains smooth muscle cells and sub-epithelial fibroblasts, including features of embryonic development that can mimic screening various intestinal disorders or degeneration.
(ii) GIO-specific shape 1-3 mm range grown on an electroconductive plate designed and developed by NGL with pay-based service to obtain ECIS base electrode with a desired signal with or without pathogens. The inventors first grew GIO on 8 well plates with an electrode. Once the GIO's electrode signal significantly differed from the media, the inventors grew the GIO on HT-scale 96 well plate with two electrodes to capture the signal better in each GIO, as shown in FIG. 20.
(iii) HT electroconductive plate of 96 well format was well adjusted by the inventors' GIO and produced a highly sensitive signal. The inventors can obtain >50 varieties of treatment with LPS or pathogen or non-pathogen or drug within 3 h, which can take in vivo 50 animal studies over 3 months with a high maintenance cost, which provides innovative insights into our technology approach.
(iv) The inventors used a nanofarads capacitance (nF) signal curve at 64000 Hz. The inventors determined that the media sample (without GIO, baseline) average signal is 3 nF compared to GIO (˜2 nF) and monolayer epithelial cells (0.5 nF), and the same measurement was also acquired by resistance (Ohms). The inventors also determined pathogen versus non-pathogenic bacteria-treated GIO in our ECIS-based sensor arrays. The electrode signal of the GIO of the invention in 64000 Hz significantly and sensitively differentiated pathogenic versus non-pathogenic bacterial responses. The unique design of the HT-scale sensor plate of the invention with optimized GIO can also produce a signal with higher specificity as the inventors can differentiate the signal pathogen from the non-pathogen. This type of unique feature added novelty to the present invention.
(v) The inventors also compared our HT electrical signal to the biological expression of TJP (Zo1) and observed a significant correlation (negative correlation) between their signals (R²=0.86). Thus, our biological signal is highly comparable to the physiological response of the human in vivo to screen for any pathogens, drugs, and toxicity, as shown in FIG. 21.

### Example 10

### High Throughput Electroconductive Sensing Arrays

To speed up pathogen detection, the inventors grew GIO on ECIS base electrode plates to identify organoid signals and differentiate pathogen versus non-pathogen electrical signals with higher sensitivity and specificity with the following scheme:

At first, the 8-well electrode plates were used to grow GIO to identify the GIO electric signal and compare it without GIO (media). The inventors observed that GIO signals differed significantly from monolayer epithelial cells or media signals. The inventors plotted the nanofarads capacitance (nF) signal curve and determined that the medium sample (without GIO, baseline) average signal is ˜3.5 nF compared to GIO (˜2.0 nF) and monolayer epithelial cells (0.5 nF). Further, the 8 well arrays grown GIO culture were treated with pathogenic and non-pathogenic bacteria and determined their respective signals. Once it was confirmed that the electrode signal of GIO with pathogen versus non-pathogenic bacteria using 8-well electrode plates, the inventors developed a 96-well plate prototype like ECIS 96 well arrays with two electrodes insertion in each of the 96-wells. Moreover, these electrodes were wider (354 μm), and no other insulating electrode around the working electrode provided sensitive signals with minimal noises compared to the 8 well arrays.

Second, the inventors grew GIO in 96-well electrode plates and treated them with pathogenic and non-pathogenic bacteria.

Third, the inventors added Cryo-GIO in 96-well plates to compare Fresh versus Cryo-GIO preserved sample electrical signals with pathogenic and non-pathogenic bacteria. It was found that GIO signals show similar trends to differentiate pathogen versus non-pathogenic bacterial signals. Finally, the inventors evaluated the pathogenic and non-pathogenic electrical signal comparison and observed significant differences using GraphPad Prism with two-way ANOVA with the Tukey multiple comparisons test.

Signal to noise: The inventors determined the capacitance (nF) and resistance (Ohms) signal with multifrequency (500-6400 Hz). The inventors only assessed the 3200-6400 Hz electrical signal, which is a more stable signal and avoids any electrical noise signal (2000 Hz) in the calculation. The inventors also used two plates, 8-well arrays, and 96-well arrays plate, to determine the electrode conductance of the GIO system. The 96-well electrode was more comprehensive in diameter (354 μm). Therefore, their electrical signal was more sensitive and bettered, differentiating the base noise level from the media.

5.2. Drug and Environmental Factor Screening

As discussed above, the GIO of the invention can be used to test the toxicity of LPS (bacterial endotoxin). This test/screening method can be similarly followed to test/screen/analyze a variety of factors and compounds, including drugs and environmental factors. The GIO of the invention, combined with intestinal microbiota, such as those of the natural human gastrointestinal system, can therefore be utilized to analyze the impact of a variety of factors on the human gastrointestinal system in a non-invasive and rapid manner.

For screening of pathogens, drugs or any other factors to determine potential effects on the human gastrointestinal system, the GIO of the invention can be provided in a useful commercial kit. Such a kit can, for example, be comprised of:

KIT ONE: Organoid in a cryopreserve vial with an organoid maintenance medium (Intestinal medium). The organoid can then be removed from the vial and combined with a gel matrix with a dome on a multi-well electroconductive plate (such as 8,16, 96, or 384-well with electrodes).

KIT TWO: Organoid already combined with a gel-matrix on an electroconductive plate and intestinal maintenance medium, ready for the use of this technology to screen drugs, toxins, and pathogens.

KIT THREE: Organoid in cryopreserve vial with an organoid maintenance medium (Intestinal medium) and gel-matrix, which can then be used with any other plate.

All publications, patent applications, patents, and other references cited herein are incorporated by reference in their entirety for the teachings relevant to the sentence and paragraph in which the reference is presented. However, the citation of any reference herein should not be construed as an admission that such reference is available as “Prior Art” to the present application.

### REFERENCES

ADDIN Mendeley Bibliography CSL BIBLIOGRAPHY 1. Findlater, A. & Bogoch, I. I. Human Mobility and the Global Spread of Infectious Diseases: A Focus on Air Travel. Trends Parasitol. 34, 772-783 (2018).
2. Wilder-Smith, A. COVID-19 in comparison with other emerging viral diseases: risk of geographic spread via travel. Trop. Dis. Travel Med. Vaccines 7, 1-11 (2021).
3. Baker, R. E. et al. Infectious disease in an era of global change. Nat. Rev. Microbiol. 20, 193-205 (2022).
4. Graves, N. S. Since January 2020 Elsevier has created a COVID-19 resource centre with free information in English and Mandarin on the novel coronavirus COVID-19. The COVID-19 resource centre is hosted on Elsevier Connect, the company's public news and information. (2020).
5. Hall, A. J. et al. Norovirus disease in the united states. Emerg. Infect. Dis. 19, 1198-1205 (2013).
6. Parrón, I. et al. A foodborne norovirus outbreak in a nursing home and spread to staff and their household contacts. Epidemiol. Infect. 147, (2019).
7. McCracken, K. W., Howell, J. C., Wells, J. M. & Spence, J. R. Generating human intestinal tissue from pluripotent stem cells in vitro. Nat. Protoc. 6, 1920-1928 (2011).
8. Jee, J. H. et al. Development of Collagen-Based 3D Matrix for Gastrointestinal Tract-Derived Organoid Culture. Stem Cells Int. 2019, (2019).
9. In, J. G. et al. Human mini-guts: New insights into intestinal physiology and host-pathogen interactions. Nat. Rev. Gastroenterol. Hepatol. 13, 633-642 (2016).
10. Finkbeiner, S. R. et al. Stem cell-derived human intestinal organoids as an infection model for rotaviruses. MBio 3, 1-6 (2012).
11. Spence, J. R. et al. Directed differentiation of human pluripotent stem cells into intestinal tissue in vitro. Nature 470, 105-110 (2011).
12. Watson, C. L. et al. An in vivo model of human small intestine using pluripotent stem cells. Nat. Med. 20, 1310-1314 (2014).
13. Dimarco, R. L. et al. Engineering of three-dimensional microenvironments to promote contractile behavior in primary intestinal organoids. Integr. Biol. (United Kingdom) 6, 127-142 (2014).
14. Wang, Q. et al. Bioengineered intestinal muscularis complexes with long-term spontaneous and periodic contractions. PLoS One 13, 1-29 (2018).
15. Yu, H. et al. The Contributions of Human Mini-Intestines to the Study of Intestinal Physiology and Pathophysiology. Annu. Rev. Physiol. 79, 291-312 (2017).
16. Okumura, R. & Takeda, K. Roles of intestinal epithelial cells in the maintenance of gut homeostasis. Exp. Mol. Med. 49, e338-8 (2017).
17. Zhu, Z. et al. Effect of berberine on LPS-induced expression of NF-κB/MAPK signalling pathway and related inflammatory cytokines in porcine intestinal epithelial cells. Innate Immun. 26, 627-634 (2020).
18. Guo, S., Al-Sadi, R., Said, H. M. & Ma, T. Y. Lipopolysaccharide causes an increase in intestinal tight junction permeability in vitro and in vivo by inducing enterocyte membrane expression and localization of TLR-4 and CD14. Am. J. Pathol. 182, 375-387 (2013).
19. Kuo, W. T. et al. LPS receptor subunits have antagonistic roles in epithelial apoptosis and colonic carcinogenesis. Cell Death Differ. 22, 1590-1604 (2015).
20. Yu, J. et al. Pro- and Antiinflammatory Cytokine Signaling: Reciprocal Antagonism Regulates Interferon-gamma Production by Human Natural Killer Cells. Immunity 24, 575-590 (2006).
21. Schroder, K., Hertzog, P. J., Ravasi, T. & Hume, D. A. Interferon-γ: an overview of signals, mechanisms and functions. J. Leukoc. Biol. 75, 163-189 (2004).
22. Chen, J. & Ivashkiv, L. B. IFN-γ abrogates endotoxin tolerance by facilitating Toll-like receptor-induced chromatin remodeling. Proc. Natl. Acad. Sci. U.S.A 107, 19438-19443 (2010).
23. Connor, T. J., Brewer, C., Kelly, J. P. & Harkin, A. Acute stress suppresses pro-inflammatory cytokines TNF-α and IL-1β independent of a catecholamine-driven increase in IL-10 production. J. Neuroimmunol. 159, 119-128 (2005).
24. Kuo, W.-T. et al. The Tight Junction Protein ZO-1 Is Dispensable for Barrier Function but Critical for Effective Mucosal Repair. Gastroenterology 161, 1924-1939 (2021).
25. Paradis, T., Begue, H., Basmaciyan, L., Dalle, F. & Bon, F. Tight junctions as a key for pathogens invasion in intestinal epithelial cells. Int. J. Mol. Sci. 22, 1-21 (2021).
26. Guan, Y. et al. Redistribution of the tight junction protein ZO-1 during physiological shedding of mouse intestinal epithelial cells. Am. J. Physiol.—Cell Physiol. 300, 1404-1414 (2011).
27. Cicchese, J. M. et al. Dynamic balance of pro- and antiinflammatory signals controls disease and limits pathology. Immunol. Rev. 285, 147-167 (2018).
28. Bardenbacher, M. et al. Permeability analyses and three dimensional imaging of interferon gamma-induced barrier disintegration in intestinal organoids. Stem Cell Res. 35, (2019).
29. Kasendra, M. et al. Development of a primary human Small Intestine-on-a-Chip using biopsy-derived organoids. Sci. Rep. 8, 1-14 (2018).
30. Frost, T. S., Jiang, L., Lynch, R. M. & Zohar, Y. Permeability of epithelial/endothelial barriers in transwells and microfluidic bilayer devices. Micromachines (2019).
31. Mason, O. U. et al. Metagenomics reveals sediment microbial community response to Deepwater Horizon oil spill. ISME J. 8, 1464-1475 (2014).
32. Fierer, N. et al. Cross-biome metagenomic analyses of soil microbial communities and their functional attributes. Proc. Natl. Acad. Sci. U.S.A 109, 21390-21395 (2012).
33. Pennington, M. R. & Van de Walle, G. R. Electric Cell-Substrate Impedance Sensing To Monitor Viral Growth and Study Cellular Responses to Infection with Alphaherpesviruses in Real Time. mSphere 2, (2017).
34. Stolwijk, J. A., Matrougui, K., Renken, C. W. & Trebak, M. Impedance analysis of GPCR-mediated changes in endothelial barrier function: overview and fundamental considerations for stable and reproducible measurements. Pflugers Arch. Eur. J. Physiol. 467, 2193-2218 (2015).
35. Begum, A. N. et al. Rapid generation of sub-type, region-specific neurons and neural networks from human pluripotent stem cell-derived neurospheres. Stem Cell Res. (2015) doi:10.1016/j.scr.2015.10.014.
36. Begum, A. N., Aguilar, J. S., Elias, L. & Hong, Y. Silver nanoparticles exhibit coating and dose-dependent neurotoxicity in glutamatergic neurons derived from human embryonic stem cells. Neurotoxicology (2016) doi:10.1016/j.neuro.2016.08.015.
37. Grosse, A. S. et al. Cell dynamics in fetal intestinal epithelium: Implications for intestinal growth and morphogenesis. Development 138, 4423-4432 (2011).
38. Jalili-Firoozinezhad, S. et al. A complex human gut microbiome cultured in an anaerobic intestine-on-a-chip. Nat. Biomed. Eng. 3, 520-531 (2019).
39. Min, S., Kim, S. & Cho, S. W. Gastrointestinal tract modeling using organoids engineered with cellular and microbiota niches. Exp. Mol. Med. 52, 227-237 (2020).
40. Grabinger, T. et al. Ex vivo culture of intestinal crypt organoids as a model system for assessing cell death induction in intestinal epithelial cells and enteropathy. Cell Death Dis. 5, e1228-10 (2014).

1. A gastrointestinal organoid which comprises:
gastrointestinal epithelial and endothelial cells;
a stratified mesenchyme surrounding said epithelial and endothelial cells and which contains muscle cells and sub-epithelial fibroblasts; and
wherein said gastrointestinal organoid exhibits fully functional contractile behavior.

2. The gastrointestinal organoid according to claim 1, wherein said muscle cells comprise smooth muscle cells, fibroblasts, and myofibroblasts.

3. The gastrointestinal organoid according to claim 1, further comprising smooth muscle cells, an enteric nervous system and interstitial cells of Cajal.

4. The gastrointestinal organoid according to claim 1, wherein said organoid expresses restricted crypts (Ki67⁺), goblet cells (MUC2⁺), Paneth cells (lysozyme⁺), endocrine cells (PDX1⁺), and enteroendocrine cells (chromogranin A⁺).

5. The gastrointestinal organoid according to claim 1, wherein the cells of said organoid are positioned on a gel-matrix dome.

6. The gastrointestinal organoid according to claim 5, wherein said gel-matrix is comprised of acylated chitosan and a gel matrix mixture.

7. The gastrointestinal organoid according to claim 6, wherein said gel matrix dome comprising said cells of said organoid are positioned in the wells of a multi-well electroconductive plate.

8. The gastrointestinal organoid according to claim 1, further comprising or interacting with a microbiome that resembles or is the same as that of an in vivo human gastrointestinal system.

9. A method for preparing a gastrointestinal organoid, comprising:
a) culturing human induced pluripotent stem cells (HiPSCs) in a first culture media to produce 3-dimensional gut spheroid cells;
b) treating said 3-dimensional spheroid cells with Activin A and generating gut spheroids;
c) treating said gut spheroid cells with Wnt3A and fibroblast growth factor 4 to produce large gut spheroid cells having a diameter of greater than about 200 μtm;
d) seeding said large gut spheroid cells on a dome-shaped gel-matrix in an intestinal media containing Rspondin, noggin and EGF; and
e) culturing said domed-shaped gel matrix comprising large gut spheroid cells to provide a complete gastrointestinal organoid.

10. The method according to claim 9, further comprising seeding said large gut spheroid cells on a dome-shaped gel matrix in wells of a multi-well plate.

11. The method according to claim 10, wherein said multi-plate well is an electroconductive multi-well plate.

12. The method according to claim 9, wherein the treatment with Activin A is initiated at Day 3 from the beginning said culturing of 3D spheroid, derived from the human induced pluripotent stem cells (HiPSCs).

13. The method according to claim 11, wherein said Wnt3A and FGF4 are added to the spheroids at Day 6 from the beginning said culturing of 3D spheroid from the human induced pluripotent stem cells (HiPSCs).

14. The method according to claim 12, wherein said Rspondin, noggin, and EGF are added to the spheroids at Day 11 from the beginning of said culturing derived from a 3D spheroid of the human induced pluripotent stem cells (HiPSCs).

15. A gastrointestinal organoid obtained by the method of claim 8.

16. A method for determining the effect of a factor of interest to the human gastrointestinal system which comprises exposing a gastrointestinal organoid of claim 7 with a factor of interest and measuring current impedance by electric cell-substrate impedance sensing.

17. A method for determining the effect of a factor to the human gastrointestinal system, which comprises:
a) exposing a first portion of gastrointestinal organoids of claim 6 comprising electrodes in said wells of said electroconductive wells with a factor of interest,
b) measuring the current or impedance of current across said electrodes in the said first portion of said gastrointestinal organoids.

18. The method according to claim 16, further comprising:
a) not exposing a second portion of said gastrointestinal organoids comprising electrodes in said wells of said electroconductive wells with the said factor of interest,
b) measuring the current or impedance of current across said electrodes in said second portion of said gastrointestinal organoids; and
c) comparing the current or impedance of current across said electrodes in the said first portion of said gastrointestinal organoids with the current or impedance of current across said electrodes in said second portion of said gastrointestinal organoids.

19. The method according to claim 17, wherein said factor is a pathogen, drug, or environmental factor.

20. The method according to claim 18, wherein the effect to be measured is the toxicity of said factor.

21. A kit comprising a vial containing the organoid of claim 1 and an intestinal organoid maintenance medium.

22. The kit according to claim 21, wherein said organoid is positioned on a gel-matrix dome.

23. The kit according to claim 21, wherein said organoid is positioned in wells of a multi-well plate.

24. The kit according to claim 22, wherein said multi-well plate is an electroconductive multi-well plate.
